(19) United States
(12) Patent Application Publication
(10) Pub. No.: US 2026/0258147 A1
(43) Pub. Date: Sep. 3, 2026

(54) HUMAN MESOTHELIN BINDERS

(71) Applicant: MERCK SHARP & DOHME LLC, Rahway, NJ (US)

(72) Inventors: Arthur FRIDMAN, Lake Ariel, PA (US); Masahisa HANDA, Berkeley, CA (US); Chung-ming HSIEH, Newton, MA (US); Sujata SINGH, Saratoga, CA (US); Sahba TABRIZIFARD, Belmont, CA (US); Sandra Isabel VANEGAS, Fremont, CA (US)

(21) Appl. No.: 18/717,904

(22) PCT Filed: Dec. 9, 2022
(86) PCT No.: PCT/US2022/081253

§ 371 Date: Jun. 7, 2024

Related U.S. Application Data
(60) Provisional application No. 63/288,162, filed on Dec. 10, 2021.

(51) Int. Cl. C07K 16/30; A61K 49/04; A61K 49/16; C07K 16/28

(52) U.S. Cl. CPC C07K 16/30; A61K 49/04; A61K 49/16; C07K 16/2818; C07K 16/2827

(57) ABSTRACT

Antibodies and antigen-binding fragments thereof that bind human mesothelin are described.

### CROSS REFERENCE

This application claims priority to U.S. Provisional Application No. 63/288,162, filed on Dec. 10, 2021, the entirety of which is incorporated herein by reference.

### SEQUENCE LISTING

This application contains a computer readable Sequence Listing which has been submitted in XML file format with this application, the entire content of which is incorporated by reference herein in its entirety. The Sequence Listing in XML file submitted with this application is entitled “14463-047-228_SEQ_LISTING.xml”, was created on Dec. 9, 2022, and is 52,002 bytes in size.

### FIELD

The present invention relates to anti-mesothelin antibodies and antigen-binding fragments thereof that can bind to human mesothelin.

### BRIEF SUMMARY OF THE INVENTION

Mesothelin is a differentiation antigen whose expression in normal human tissues is limited to mesothelial cells lining the pleura, pericardium, and peritoneum (Chang & Pastan, Proc. Natl. Acad. Sci. USA 93:136-140 (1996); Chang et al., Int. J. Cancer 50:373-381 (1992)). However, mesothelin is highly expressed in several human cancers, including virtually all mesotheliomas and pancreatic adenocarcinomas, and approximately 70% of ovarian cancers and 50% of lung adenocarcinomas (Ordonez, Mod Pathol. 16:192-197 (2003); Argani et al., Clin. Cancer Res. 7:3862-3868 (2001); Hassan et al., Appl. Immunohistochem Mol. Morphol. 13:243-247 (2005); Ordonez, Am. J. Surg. Pathol. 27:1418-1428 (2003)).

The mesothelin gene encodes a precursor protein of 71 kDa that is processed to a 31 kDa shed protein called megakaryocyte potentiating factor (MPF) and a 40 kDa fragment, mesothelin, that is attached to the cell membrane by a glycosyl-phosphatidylinositol (GPI) anchor. MPF was isolated from the culture supernatant of a pancreatic cancer cell line and was so named because it stimulated the megakaryocyte colony-forming activity of interleukin-3 in mouse bone marrow cultures. The biologic function of mesothelin is not known; however, results of recent studies suggest that mesothelin may play a role in ovarian cancer metastasis by binding to MUC16/CA-125 (Rump et al., J. Biol. Chem. 279:9190-9198 (2004)). A small amount of cell bound mesothelin is shed into the serum and has been shown to be elevated in patients with mesothelioma and ovarian cancer (Hassan et al., Clin. Cancer Res. 12:447-453 (2006)).

Mesothelin is a promising candidate for tumor-specific therapy given its limited expression in normal tissues and high expression in several cancers. These therapies include agents that target cell surface mesothelin or elicit an immune response against mesothelin. Agents that are in the clinic or about to enter clinical trials include CAT-5001, MORAb-009, and CRS-207 (Hassan & Ho, Eur. J. Cancer 44:46-53 (2008).

Therapeutic antibodies that bind to mesothelin may be useful for the treatment of cancer. Monoclonal antibodies (mAbs) both in monotherapy and in combination regiments has emerged as one of the fastest growing and most effective therapeutic strategies for the treatment of solid tumors and hematological diseases. Between 2015 and 2017, the U.S. Food and Drug Administration approved 27 therapeutic mAbs (Tsumoto et al., Immunotherapy 11:119-127 (2019)) increasing the total of clinically used mAbs and biosimilars in 2017 to 57 and 11, respectively (Grilo & Mantalaris, Trends Biotechnol. 37:9-16 (2019)). As of late 2019, numerous companies were supporting over 550 novel antibody therapeutics in early phase clinical trials, with approximately half of these against oncology targets (Kaplon et al., MAbs 12:1-24 (2020)).

The present invention provides antibody and antigen-binding fragments thereof that specifically bind to human mesothelin (human MSLN) comprising the amino acid sequence set forth in SEQ ID NO: 1. These human MSLN binders display weak or no detectable binding to non-human primate mesothelin and may be useful in treatments for cancers and proliferative diseases.

The human MSLN binders of the present invention comprises six complementarity determining regions (CDRs) of an antibody comprising (a) a heavy chain (HC) variable domain (V_{H}) comprising the amino acid sequence set forth in SEQ ID NO: 2, 26, 27, or 28 and a light chain (LC) variable domain (V_{L}) comprising the amino acid sequence set forth in SEQ ID NO: 3; (b) a V_{H} comprising the amino acid sequence set forth in SEQ ID NO: 10 and a V_{L} comprising the amino acid sequence set forth in SEQ ID NO: 11; or, (c) a V_{H} comprising the amino acid sequence set forth in SEQ ID NO: 18 and a V_{L} comprising the amino acid sequence set forth in SEQ ID NO: 19. The CDR sequences of the human MSLN binder may be defined according to any numbering scheme useful for defining CDR sequences including but not limited to the Kabat, Chothia, AbM, ImMunoGeneTics (IMGT), or Contact numbering scheme. In a particular embodiment, the CDRs are defined by Kabat or IMGT. In a particular embodiment, the CDRs are defined by Kabat.

In a further embodiment of the human MSLN binder, the V_{H} comprises a CDR 1 comprising the amino acid sequence set forth in SEQ ID NO: 4, a CDR 2 comprising the amino acid sequence set forth in SEQ ID NO: 5, and a CDR 3 comprising the amino acid sequence set forth in SEQ ID NO:6; and, the V_{L} comprises a CDR 1 comprising the amino acid sequence set forth in SEQ ID NO: 7, a CDR 2 comprising the amino acid sequence set forth in SEQ ID NO: 8, and a CDR 3 comprising the amino acid sequence set forth in SEQ ID NO: 9.

In a further embodiment of the human MSLN binder, the V_{H} comprises a CDR 1 comprising the amino acid sequence set forth in SEQ ID NO: 12, a CDR 2 comprising the amino acid sequence set forth in SEQ ID NO: 13, and a CDR 3 comprising the amino acid sequence set forth in SEQ ID NO:14; and, the V_{L} comprises a CDR 1 comprising the amino acid sequence set forth in SEQ ID NO: 15, a CDR 2 comprising the amino acid sequence set forth in SEQ ID NO: 16, and a CDR 3 comprising the amino acid sequence set forth in SEQ ID NO: 17.

In a further embodiment of the human MSLN binder, the V_{H} comprises a CDR 1 comprising the amino acid sequence set forth in SEQ ID NO: 20, a CDR 2 comprising the amino acid sequence set forth in SEQ ID NO: 21, and a CDR 3 comprising the amino acid sequence set forth in SEQ ID NO:22; and, the V_{L} comprises a CDR 1 comprising the amino acid sequence set forth in SEQ ID NO: 23, a CDR 2 comprising the amino acid sequence set forth in SEQ ID NO: 24, and a CDR 3 comprising the amino acid sequence set forth in SEQ ID NO: 25.

In a further embodiment, the human MSLN binder comprises a V_{H} comprising (i) the amino acid sequence set forth in SEQ ID NO: 2 or an amino acid sequence having the 2nd amino acid residue to the 116th amino acid residue of the amino acid sequence ranging from the 2nd amino acid residue to the 116th amino acid residue set forth in SEQ ID NO: 2 and the 1 st amino acid residue of the amino acid sequence being pyroglutamate; (ii) the amino acid sequence set forth in SEQ ID NO: 26 or an amino acid sequence having the 2nd amino acid residue to the 116th amino acid residue of the amino acid sequence ranging from the 2nd amino acid residue to the 116th amino acid residue set forth in SEQ ID NO: 26 and the 1st amino acid residue of the amino acid sequence being pyroglutamate; (iii) the amino acid sequence set forth in SEQ ID NO: 27 or an amino acid sequence having the 2nd amino acid residue to the 116th amino acid residue of the amino acid sequence ranging from the 2nd amino acid residue to the 116th amino acid residue set forth in SEQ ID NO: 27 and the 1st amino acid residue of the amino acid sequence being pyroglutamate; or (iii) the amino acid sequence set forth in SEQ ID NO: 28 or an amino acid sequence having the 2nd amino acid residue to the 116th amino acid residue of the amino acid sequence ranging from the 2nd amino acid residue to the 116th amino acid residue set forth in SEQ ID NO: 28 and the 1st amino acid residue of the amino acid sequence being pyroglutamate, and a V_{L} comprising the amino acid sequence set forth in SEQ ID NO: 3. In a further embodiment, the human MSLN binder comprises a V_{H} comprising the amino acid sequence set forth in SEQ ID NO: 2, SEQ ID NO: 26, SEQ ID NO: 27, or SEQ ID NO: 28 and a V_{L} comprising the amino acid sequence set forth in SEQ ID NO: 3.

In a further embodiment, the human MSLN binder comprises a V_{H} comprising the amino acid sequence set forth in SEQ ID NO: 10 or an amino acid sequence having the 2nd amino acid residue to the 116th amino acid residue of the amino acid sequence ranging from the 2nd amino acid residue to the 116th amino acid residue set forth in SEQ ID NO: 10 and the 1st amino acid residue of the amino acid sequence being pyroglutamate, and a V_{L} comprising the amino acid sequence set forth in SEQ ID NO: 11 or an amino acid sequence having the 2nd amino acid residue to the 107th amino acid residue of the amino acid sequence ranging from the 2nd amino acid residue to the 107th amino acid residue set forth in SEQ ID NO: 11 and the 1st amino acid residue of the amino acid sequence being pyroglutamate. In a further embodiment, the human MSLN binder comprises a V_{H} comprising the amino acid sequence set forth in SEQ ID NO: 10 and a V_{L} comprising the amino acid sequence set forth in SEQ ID NO: 11.

In a further embodiment, the human MSLN binder comprises a V_{H} comprising the amino acid sequence set forth in SEQ ID NO: 18 or an amino acid sequence having the 2nd amino acid residue to the 118th amino acid residue of the amino acid sequence ranging from the 2nd amino acid residue to the 118th amino acid residue set forth in SEQ ID NO: 18 and the 1st amino acid residue of the amino acid sequence being pyroglutamate, and a V_{L} comprising the amino acid sequence set forth in SEQ ID NO: 19 or an amino acid sequence having the 2nd amino acid residue to the 108th amino acid residue of the amino acid sequence ranging from the 2nd amino acid residue to the 108th amino acid residue set forth in SEQ ID NO: 19 and the 1st amino acid residue of the amino acid sequence being pyroglutamate. In a further embodiment, the human MSLN binder comprises a V_{H} comprising the amino acid sequence set forth in SEQ ID NO: 18 and a V_{L} comprising the amino acid sequence set forth in SEQ ID NO: 19.

The present invention further provides a human MSLN binder comprising a V_{H} comprising an amino acid sequence with at least 90% identity to the amino acid sequence set forth in SEQ ID NO: 2, SEQ ID NO: 26, SEQ ID NO: 27, or SEQ ID NO: 28; and a V_{L} comprising an amino acid sequence with at least 90% identity to the amino acid sequence set forth in SEQ ID NO: 3, with the proviso that the V_{H} comprises a CDR 1 comprising the amino acid sequence set forth in SEQ ID NO: 4, a CDR 2 comprising the amino acid sequence set forth in SEQ ID NO: 5, and a CDR 3 comprising the amino acid sequence set forth in SEQ ID NO:6; and, the V_{L} comprises a CDR 1 comprising the amino acid sequence set forth in SEQ ID NO: 7, a CDR 2 comprising the amino acid sequence set forth in SEQ ID NO: 8, and a CDR 3 comprising the amino acid sequence set forth in SEQ ID NO: 9.

The present invention further provides a human MSLN binder comprising a V_{H} comprising an amino acid sequence with at least 90% identity to the amino acid sequence set forth in SEQ ID NO: 10; and a V_{L} comprising an amino acid sequence with at least 90% identity to the amino acid sequence set forth in SEQ ID NO: 11.

The present invention further provides a human MSLN binder comprising a V_{H} comprising an amino acid sequence with at least 90% identity to the amino acid sequence set forth in SEQ ID NO: 10; and a V_{L} comprising an amino acid sequence with at least 90% identity to the amino acid sequence set forth in SEQ ID NO: 11, with the proviso that the V_{H} comprises a CDR 1 comprising the amino acid sequence set forth in SEQ ID NO: 12, a CDR 2 comprising the amino acid sequence set forth in SEQ ID NO: 13, and a CDR 3 comprising the amino acid sequence set forth in SEQ ID NO: 14; and, the V_{L} comprises a CDR 1 comprising the amino acid sequence set forth in SEQ ID NO: 15, a CDR 2 comprising the amino acid sequence set forth in SEQ ID NO: 16, and a CDR 3 comprising the amino acid sequence set forth in SEQ ID NO: 17.

The present invention further provides a human MSLN binder comprising a V_{H} comprising an amino acid sequence with at least 90% identity to the amino acid sequence set forth in SEQ ID NO: 18; and a V_{L} comprising an amino acid sequence with at least 90% identity to the amino acid sequence set forth in SEQ ID NO: 19.

The present invention further provides a human MSLN binder comprising a V_{H} comprising an amino acid sequence with at least 90% identity to the amino acid sequence set forth in SEQ ID NO: 18; and a V_{L} comprising an amino acid sequence with at least 90% identity to the amino acid sequence set forth in SEQ ID NO: 19, with the proviso the V_{H} comprises a CDR 1 comprising the amino acid sequence set forth in SEQ ID NO: 20, a CDR 2 comprising the amino acid sequence set forth in SEQ ID NO: 21, and a CDR 3 comprising the amino acid sequence set forth in SEQ ID NO:22; and, the V_{L} comprises a CDR 1 comprising the amino acid sequence set forth in SEQ ID NO: 23, a CDR 2 comprising the amino acid sequence set forth in SEQ ID NO: 24, and a CDR 3 comprising the amino acid sequence set forth in SEQ ID NO: 25.

In a further embodiment, the human MSLN binder is an antibody that comprises a heavy chain constant domain of the IgG1 isotype. In particular embodiments, the heavy chain constant domain comprises the amino acid sequence set forth in SEQ ID NO: 29. In particular embodiments, the heavy chain constant domain comprises an amino acid sequence having the 1st amino acid residue to the 329th amino acid residue of the amino acid sequence ranging from the 1st amino acid residue to the 329th amino acid residue set forth in SEQ ID NO: 29. In a further embodiment, the heavy chain constant domain of the IgG1 isotype comprises an Fc domain comprising one or more mutations that render the constant domain effector-silent. In particular embodiments the effector-silent constant domain comprises an amino acid sequence set forth in SEQ ID NO: 30, SEQ ID NO: 31, SEQ ID NO: 32, SEQ ID NO: 33, SEQ ID NO: 34, SEQ ID NO: 35, SEQ ID NO: 36, or SEQ ID NO: 37. In particular embodiments, the effector-silent constant domain comprises the amino acid sequence set forth in SEQ ID NO: 30 or an amino acid sequence having the 1st amino acid residue to the 329th amino acid residue of the amino acid sequence ranging from the 1st amino acid residue to the 329th amino acid residue set forth in SEQ ID NO: 30. In particular embodiments the effector-silent constant domain comprises the amino acid sequence set forth in SEQ ID NO: 31 or an amino acid sequence having the 1st amino acid residue to the 329th amino acid residue of the amino acid sequence ranging from the 1st amino acid residue to the 329th amino acid residue set forth in SEQ ID NO: 31. In particular embodiments the effector-silent constant domain comprises the amino acid sequence set forth in SEQ ID NO: 32 or an amino acid sequence having the 1st amino acid residue to the 329th amino acid residue of the amino acid sequence ranging from the 1st amino acid residue to the 329th amino acid residue set forth in SEQ ID NO: 32. In particular embodiments the effector-silent constant domain comprises the amino acid sequence set forth in SEQ ID NO: 33 or an amino acid sequence having the 1st amino acid residue to the 329th amino acid residue of the amino acid sequence ranging from the 1st amino acid residue to the 329th amino acid residue set forth in SEQ ID NO: 33. In particular embodiments the effector-silent constant domain comprises the amino acid sequence set forth in SEQ ID NO: 34 or an amino acid sequence having the 1st amino acid residue to the 329th amino acid residue of the amino acid sequence ranging from the 1st amino acid residue to the 329th amino acid residue set forth in SEQ ID NO: 34. In particular embodiments the effector-silent constant domain comprises the amino acid sequence set forth in SEQ ID NO: 35 or an amino acid sequence having the 1st amino acid residue to the 329th amino acid residue of the amino acid sequence ranging from the 1st amino acid residue to the 329th amino acid residue set forth in SEQ ID NO: 35. In particular embodiments the effector-silent constant domain comprises the amino acid sequence set forth in SEQ ID NO: 36 or an amino acid sequence having the 1st amino acid residue to the 329th amino acid residue of the amino acid sequence ranging from the 1st amino acid residue to the 329th amino acid residue set forth in SEQ ID NO: 36. In particular embodiments the effector-silent constant domain comprises the amino acid sequence set forth in SEQ ID NO: 37 or an amino acid sequence having the 1st amino acid residue to the 329th amino acid residue of the amino acid sequence ranging from the 1st amino acid residue to the 329th amino acid residue set forth in SEQ ID NO: 37. In a particular embodiment, the constant domain comprises the amino acid sequence set forth in SEQ ID NO: 31 or an amino acid sequence having the 1st amino acid residue to the 329th amino acid residue of the amino acid sequence ranging from the 1st amino acid residue to the 329th amino acid residue set forth in SEQ ID NO: 31.

In a further embodiment, the light chain may comprise a human kappa light chain constant domain or a lambda light chain constant domain. In further embodiments disclosed herein, the light chain constant domain may comprise a human kappa light chain constant domain comprising the amino acid sequence set forth in SEQ ID NO: 38.

The present invention further provides a human MSLN binder comprising a Fab fragment, a Fab′ fragment, a F(ab′)₂ fragment, an Fv region, or an ScFv. In particular embodiments, a bispecific antibody is provided wherein one arm of the bispecific antibody comprises a human MSLN binder selected from the group consisting of Fab′ fragment or ScFv and the other arm of the bispecific antigen comprises a Fab′ or ScFv specific for an antigen other than human MSLN.

The present disclosure further provides a human MSLN binder, wherein the human MSLN binder comprises one or two of the modifications relative to the human MSLN binder provided herein selected from (i) a C-terminal amino acid residue K of a heavy chain being removed; and (ii) a N-terminal amino acid residue E or Q of a VH, a VL, a heavy chain or a light chain being substituted with pyroglutamate.

The present invention further comprises a composition comprising the human MSLN binder disclosed herein and a pharmaceutically acceptable carrier or diluent.

The present invention further provides a method for treating cancer or proliferative disease in an individual in need of the treatment comprising administering to the individual a therapeutically effective amount of a human MSLN binder disclosed herein or a composition disclosed herein to treat the cancer or a proliferative disease.

The present invention further provides a human MSLN binder or composition disclosed herein for treatment of cancers or proliferative diseases.

The present invention further provides for the use of a human MSLN binder disclosed herein for the manufacture of a medicament for treating cancer or proliferative disease.

The present invention further provides a combination therapy for treating a cancer or proliferative disease comprising a human MSLN binder or composition disclosed herein and a therapeutic agent. In a further embodiment, the therapeutic agent is a chemotherapy agent or a therapeutic antibody. In a further still embodiment, the antibody is an anti-PD1 or anti-PD-L1 antibody.

The present invention further provides a nucleic acid molecule encoding the V_{H} of a human MSLN binder disclosed herein and/or VL of a human MSLN binder disclosed herein. Further provided is an expression vector comprising one or more of the nucleic acid molecules disclosed herein. Further provided is a host cell comprising an expression vector comprising one or more of the nucleic acid molecules disclosed herein.

The present invention further provides a method for producing a human MSLN binder comprising (a) providing a host cell comprising an expression vector comprising one or more of the nucleic acid molecules disclosed herein; (b) cultivating the host cell in a medium under conditions suitable for expressing the human MSLN binder; and (c) isolating the human MSLN binder from the medium.

The present disclosure further provides a human MSLN binder obtainable by expressing the nucleic acid or the expression vector provided herein in a host cell.

The present invention further provides a human MSLN binder disclosed herein conjugated to a detectable moiety.

The present invention further provides a method for detecting human MSLN on the surface of a cell in a patient comprising administering to the patient a human MSLN binder disclosed herein conjugated to a detectable moiety and detecting the cells in the patient bound to the human MSLN binder conjugated to the detectable moiety. In particular embodiments, the detectable moiety is detectable by magnetic resonance imaging (MRI) or by X-ray imaging.

The present invention further provides a cell that expresses a human MSLN binder on the cell surface. The human MSLN binder that is expressed may be a whole antibody, bispecific antibody, a Fab′ fragment, an ScFv, or a tandem ScFv. In particular embodiments, the cell that expresses the human MSLN binder is an immune cell, for example a T cell or an NK cell.

The present invention further provides a human MSLN binder that is conjugated to a cytotoxin. The human MSLN binder may be whole antibody, bispecific antibody, a Fab′ fragment, or an ScFv.

### BRIEF DESCRIPTION OF THE DRAWINGS

FIG. 1 shows ELISA binding of the SV017.47D7.1C2 antibody, as well as the positive control MORAB-009, to the soluble and membrane-bound human MSLN. Similar to the MORAB-009, SV017.47D7.1C2 binds to both soluble and membrane-bound human MSLN. HuMSLN refers to human MSLN and CHOK1 refers to Chinese hamster ovary K1 cells.

FIG. 2 shows ELISA binding of the SV018.20B12.1D1 antibody, as well as the positive control MORAB-009 to the soluble and membrane-bound human MSLN. SV018.20B12.1D1 binds to soluble human MSLN with lower potency than it does to membrane-bound human MSLN. HuMSLN refers to human MSLN and CHOK1 refers to Chinese hamster ovary K1 cells.

FIG. 3 shows ELISA binding of the SV018.45B6.1G1 antibody, as well as the positive control MORAB-009 to the soluble and membrane-bound human MSLN. SV018.45B6.1G1 binds to soluble human MSLN with lower potency than it does to membrane-bound human MSLN. HuMSLN refers to human MSLN and CHOK1 refers to Chinese hamster ovary K1 cells.

FIG. 4 shows a cartoon showing human MSLN generated from a ~70 kDa precursor protein by endoprotease furin cleavage to release the N-terminal region MPF (megakaryocyte potentiating factor) and the 40 kDa membrane bound mesothelin (GPI linked to the cell surface).

### DETAILED DESCRIPTION OF THE INVENTION

### Definitions

So that the invention may be more readily understood, certain technical and scientific terms are specifically defined below. Unless specifically defined elsewhere in this document, all other technical and scientific terms used herein have the meaning commonly understood by one of ordinary skill in the art to which this invention belongs.

As used herein, including the appended claims, the singular forms of words such as “a,” “an,” and “the,” include their corresponding plural references unless the context clearly dictates otherwise.

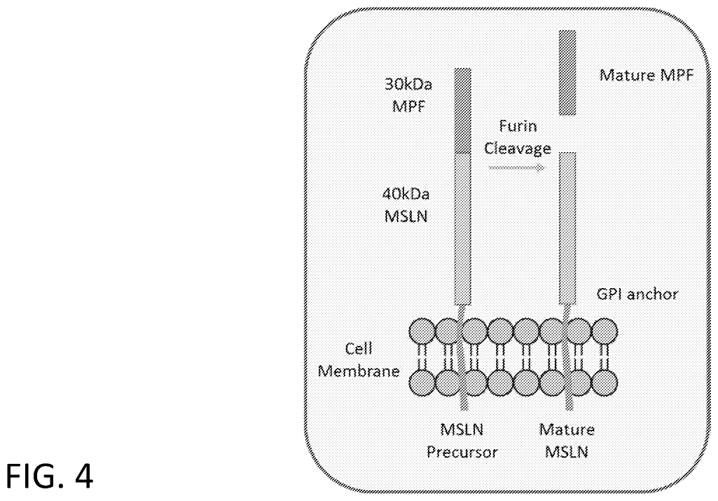

As used herein, the term “mesothelin” or “MSLN” refers to a differentiation antigen whose expression in normal human tissues is limited to mesothelial cells lining the pleura, pericardium and peritoneum. MSLN is generated from a ~70 kDa precursor protein that is cleaved by endoprotease furin to release the N-terminal region MPF (megakaryocyte potentiating factor) and 40 kDa membrane bound mesothelin (GPI linked to the cell surface) (See FIG. 4). The amino acid sequence of human mesothelin precursor is set forth in SEQ ID NO: 1. The mature form comprises amino acids 296-622.

As used herein, the term “human MSLN” refers to the mature form of human mesothelin and the term “rhesus MSLN” refers to the mature form of Rhesus (Macaca mulatta) mesothelin. The amino acid sequences for the precursor and mature forms of rhesus MSLN are shown in SEQ ID NO: 39.

As used herein, the term “human MSLN binder” refers to an antibody or antigen binding fragment thereof that binds to soluble and/or membrane-bound human MSLN. A human MSLN binder includes but is not limited to a bivalent antibody tetramer (2H+2L), a monovalent antibody (H+L), a bi-specific antibody that targets human MSLN and another target, a Fab fragment, a Fab′ fragment, a F(ab′)₂ fragment, an Fv region, and an ScFv.

As used herein, the term “affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (KD). Affinity can be measured by common methods known in the art, including KinExA and surface plasmon resonance (SPR; Biacore™). Specific illustrative and exemplary embodiments for measuring binding affinity are described in the following.

As used herein, the term “administration” and “treatment,” as it applies to an animal, human, experimental subject, cell, tissue, organ, or biological fluid, refers to contact of an exogenous pharmaceutical, therapeutic, diagnostic agent, or composition comprising a human MSLN binder as disclosed herein to the animal, human, subject, cell, tissue, organ, or biological fluid. Treatment of a cell encompasses contact of a reagent to the cell, as well as contact of a reagent to a fluid, where the fluid is in contact with the cell. “Administration” and “treatment” also means in vitro and ex vivo treatments, e.g., of a cell, by a reagent, diagnostic, binding compound, or by another cell. The term “subject” includes any organism, preferably an animal, more preferably a mammal (e.g., human, rat, mouse, dog, cat, rabbit). In a preferred embodiment, the term “subject” refers to a human.

As used herein, the term “amino acid” refers to a simple organic compound containing both a carboxyl (—COOH) and an amino (—NH₂) group. Amino acids are the building blocks for proteins, polypeptides, and peptides. Amino acids occur in L-form and D-form, with the L-form in naturally occurring proteins, polypeptides, and peptides. Amino acids and their code names are set forth in the following chart.

| | Three letter | One letter |
|---|---|---|
| Amino acid | code | code |
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

As used herein, the term “antibody” or “immunoglobulin” as used herein refers to a glycoprotein comprising at least two heavy chains (HCs) and two light chains (LCs) inter-connected by disulfide bonds. Each HC is comprised of a heavy chain variable region or domain (V_{H}) and a heavy chain constant region or domain. Each light chain is comprised of an LC variable region or domain (V_{L}) and a LC constant domain. In certain naturally occurring IgG, IgD and IgA antibodies, the heavy chain constant region is comprised of three domains, CH1, CH2 and CH3. In general, the basic antibody structural unit for antibodies is a Y-shaped tetramer comprising two HC/LC pairs (2H). Each tetramer includes two identical pairs of polypeptide chains, each pair having one LC (about 25 kDa) and HC chain (about 50-70 kDa) (H+L). Each HC: LC pair comprises one V_{H}:one V_{L} pair. The one V_{H}:one V_{L} pair may be referred to by the term “Fab”. Thus, each antibody tetramer comprises two Fabs, one per each arm of the Y-shaped antibody. In certain embodiments, the antibody may include post-translational modifications thereof (e.g., C-terminal Lysine clipping in the heavy chain, conversion of glutamine or glutamic acid to pyroglutamate) which may occur when recombinantly expressed in host cells (e.g., CHO cells), or during purification/storage.

The LC constant domain is comprised of one domain, CL. The human V_{H} includes seven family members: V_{H}1, V_{H}2, V_{H}3, V_{H}4, V_{H}5, V_{H}6, and V_{H}7; and the human V_{L} includes 16 family members: V_{κ}1, V_{κ}2, V_{κ}3, V_{κ}4, V_{κ}5, V_{κ}6, V_{λ}1, V_{λ}2, V_{λ}3, V_{λ}4, V_{λ}5, V_{λ}6, V_{λ}7, V_{λ}8, V_{λ}9, and V_{λ}10. Each of these family members can be further divided into particular subtypes. The V_{H} and V_{L} can be further subdivided into regions of hypervariability, termed complementarity determining region (CDR) areas, interspersed with regions that are more conserved, termed framework regions (FR). Each V_{H} and V_{L} is composed of three CDR regions and four FR regions, arranged from amino-terminus to carboxy-terminus in the following order: FR1, CDR1, FR2, CDR2, FR3, CDR3, FR4. Numbering of the amino acids in a VH may be determined using the Kabat numbering scheme. See Béranger, et al., Ed. Ginetoux, Correspondence between the IMGT unique numbering for C-DOMAIN, the IMGT exon numbering, the Eu and Kabat numberings: Human IGHG, Created: 17/05/2001, Version: Aug. 6, 2016, which is accessible at www.imgt.org/IMGTScientificChart/Numbering/Hu_IGHGnber.html).

The constant regions of the antibodies may mediate the binding of the immunoglobulin to host tissues or factors, including various cells of the immune system (e.g., effector cells) and the first component (C1q) of the classical complement system. Typically, the numbering of the amino acids in the heavy chain constant domain begins with number 118, which is in accordance with the Eu numbering scheme. The Eu numbering scheme is based upon the amino acid sequence of human IgG1 (Eu), which has a constant domain that begins at amino acid position 118 of the amino acid sequence of the IgG1 described in Edelman et al., Proc. Natl. Acad. Sci. USA. 63:78-85 (1969), and is shown for the IgG1, IgG2, IgG3, and IgG4 constant domains in Béranger et al., op. cit.

The variable regions of the heavy and light chains contain a binding domain comprising the CDRs that interacts with an antigen. A number of methods are available in the art for defining CDR sequences of antibody variable domains (see Dondelinger et al., Frontiers in Immunol. 9: Article 2278 (2018)). The common numbering schemes include the following.

Kabat numbering scheme is based on sequence variability and is the most commonly used (See Kabat et al. Sequences of Proteins of Immunological Interest, 5th Ed. Public Health Service, National Institutes of Health, Bethesda, Md. (1991) (defining the CDR regions of an antibody by sequence). Chothia numbering scheme is based on the location of the structural loop region (See Chothia & Lesk, J. Mol. Biol. 196:901-917 (1987); Al-Lazikani et al., J. Mol. Biol. 273:927-948 (1997)). AbM numbering scheme is a compromise between the two used by Oxford Molecular's AbM antibody modelling software (see Karu et al, ILAR Journal 37:132-141 (1995). Contact numbering scheme is based on an analysis of the available complex crystal structures (See www.bioinf.org.uk: Prof. Andrew C. R. Martin's Group; Abhinandan & Martin, Mol. Immunol. 45:3832-3839 (2008)). IMGT (ImMunoGeneTics) numbering scheme is a standardized numbering system for all the protein sequences of the immunoglobulin superfamily, including variable domains from antibody light and heavy chains as well as T cell receptor chains from different species and counts residues continuously from 1 to 128 based on the germ-line V sequence alignment (see Giudicelli et 5 al., Nucleic Acids Res. 25:206-11 (1997); Lefranc, Immunol Today 18: 509 (1997); Lefranc et al., Dev Comp Immunol. 27:55-77 (2003)).

The following general rules disclosed in www.bioinf.org.uk: Prof. Andrew C. R. Martin's Group and reproduced in Table 1 below may be used to define the CDRs in an antibody sequence that includes those amino acids that specifically interact with the amino acids comprising the epitope in the antigen to which the antibody binds. There are rare examples where these generally constant features do not occur; however, the Cys residues are the most conserved feature.

TABLE 1

| Loop | Kabat | AbM | Chothia¹ | Contact² | IMGT |
|---|---|---|---|---|---|
| L1 | L24--L34 | L24--L34 | L24--L34 | L30--L36 | L27--L32 |
| L2 | L50--L56 | L50--L56 | L50--L56 | L46--L55 | L50--L52 |
| L3 | L89--L97 | L89--L97 | L89--L97 | L89--L96 | L89--L97 |
| H1 | H31--H35B | H26--H35B | H26--H32 . . . 34 | H30--H35B | H26--H35B |
| | (Kabat Numbering)³ | | | | |
| H1 | H31--H35 | H26--H35 | H26--H32 | H30--H35 | H26--H33 |
| | (Chothia Numbering) | | | | |
| H2 | H50--H65 | H50--H58 | H52--H56 | H47--H58 | H51--H56 |
| H3 | H95--H102 | H95--H102 | H95--H102 | H93--H101 | H93--H102 |

¹Some of these numbering schemes (particularly for Chothia loops) vary depending on the individual publication examined.
²Any of the numbering schemes can be used for these CDR definitions, except the Contact numbering scheme uses the Chothia or Martin (Enhanced Chothia) definition.
³The end of the Chothia CDR-H1 loop when numbered using the Kabat numbering convention varies between H32 and H34 depending on the length of the loop. (This is because the Kabat numbering scheme places the insertions at H35A and H35B.)
If neither H35A nor H35B is present, the loop ends at H32
If only H35A is present, the loop ends at H33
If both H35A and H35B are present, the loop ends at H34

The entire nucleotide sequence of the heavy chain and light chain variable regions are commonly numbered according to Kabat while the three CDRs within the variable region may be defined according to any one of the aforementioned numbering schemes.

In general, the state of the art recognizes that in many cases, the CDR3 region of the heavy chain is the primary determinant of antibody specificity, and examples of specific antibody generation based on CDR3 of the heavy chain alone are known in the art (e.g., Beiboer et al., J. Mol. Biol. 296:833-849 (2000); Klimka et al., British J. Cancer 83:252-260 (2000); Rader et al., Proc. Natl. Acad. Sci. USA 95:8910-8915 (1998); Xu et al., Immunity As used herein, the term “Fc domain”, or “Fc” as used herein is the 13:37-45 (2000). crystallizable fragment domain or region obtained from an antibody that comprises the CH2 and CH3 domains of an antibody. In an antibody, the two Fc domains are held together by two or more disulfide bonds and by hydrophobic interactions of the CH3 domains. The Fc domain may be obtained by digesting an antibody with the protease papain. Typically, amino acids in the Fc domain are numbered according to the Eu numbering convention (See Edelmann et al., Biochem. 63:78-85 (1969)).

As used herein, the term “antigen” as used herein refers to any foreign substance which induces an immune response in the body.

As used herein, the term “antigen binding fragment” refers to a polypeptide or polypeptides comprising a fragment of a full-length antibody, which retains the ability to specifically bind to the antigen bound by the full length antibody, and/or to compete with the full length antibody for specifically binding to the antigen. Examples of antigen binding fragments include but are not limited to Fab fragment, Fab′ fragment, F(ab′)2 fragment, Fv region, and scFv.

As used herein, the term “Fab fragment” refers to an antigen binder comprising one antibody light chain and the CH1 and V_{H} of one antibody heavy chain. The heavy chain of a Fab molecule cannot form a disulfide bond with another heavy chain molecule. A “Fab fragment” can be the product of papain cleavage of an antibody.

As used herein, the term “Fab′ fragment” refers to an antigen binder comprising one antibody light chain and a portion or fragment of one antibody heavy chain that contains the V_{H} and the CH1 domain up to a region between the CH1 and CH2 domains, such that an interchain disulfide bond can be formed between the two heavy chains of two Fab′ fragments to form a F(ab′)₂ molecule.

As used herein, the term “F(ab′)2 fragment” refers to an antigen binder comprising two antibody light chains and two heavy chains containing the V_{H} and the CH1 domain up to a region between the CH1 and CH2 domains, such that an interchain disulfide bond is formed between the two heavy chains. An F(ab′)₂ fragment thus is composed of two Fab′ fragments that are held together by a disulfide bond between the two heavy chains. An “F(ab′)₂ fragment” can be the product of pepsin cleavage of an antibody.

As used herein, the term “Fv region” refers to an antigen binder comprising the variable regions from both the heavy and light chains of an antibody but lacks the constant regions.

As used herein, the term “ScFv” or “single-chain variable fragment” refers to a fusion protein comprising a V_{H} and V_{L} fused or linked together by a short linker peptide of ten to about 25 amino acids. The linker is usually rich in glycine for flexibility, as well as serine or threonine for solubility, and can either connect the N-terminus of the V_{H} with the C-terminus of the V_{L}, or vice versa. This protein retains the specificity of the original immunoglobulin, despite removal of the constant regions and the introduction of the linker.

As used herein, the term “diabody” refers to an antigen binder comprising a small antibody fragment with two antigen-binding regions, which fragments comprise a heavy chain variable domain (V_{H}) connected to a light chain variable domain (V_{L}) in the same polypeptide chain (V_{H}-V_{L} or V_{L}-V_{H}). By using a linker that is too short to allow pairing between the two domains on the same chain, the domains are forced to pair with the complementarity domains of another chain and create two antigen-binding regions. Diabodies are described more fully in, e.g., EP 404,097; WO 93/11161; and Holliger et al. (1993) Proc. Natl. Acad. Sci. USA 90:6444-6448. For a review of engineered antibody variants generally see Holliger and Hudson (2005) Nat. Biotechnol. 23:1126-1136.

These and other potential constructs are described at Chan & Carter (2010) Nat. Rev. Immunol. 10:301. These antibody fragments are obtained using conventional techniques known to those with skill in the art, and the fragments are screened for utility in the same manner as are intact antibodies. Antigen-binding fragments can be produced by recombinant DNA techniques, or by enzymatic or chemical cleavage of intact immunoglobulins.

As used herein, the term “isolated” antibodies or antigen-binding fragments thereof are at least partially free of other biological molecules from the cells or cell cultures in which they are produced. Such biological molecules include nucleic acids, proteins, lipids, carbohydrates, or other material such as cellular debris and growth medium. An isolated antibody or antigen-binding fragment may further be at least partially free of expression system components such as biological molecules from a host cell or of the growth medium thereof. Generally, the term “isolated” is not intended to refer to a complete absence of such biological molecules or to an absence of water, buffers, or salts or to components of a pharmaceutical formulation that includes the antibodies or fragments.

As used herein, the term “monoclonal antibody” refers to a population of substantially homogeneous antibodies, i.e., the antibody molecules comprising the population are identical in amino acid sequence except for possible naturally occurring mutations that may be present in minor amounts. In contrast, conventional (polyclonal) antibody preparations typically include a multitude of different antibodies having different amino acid sequences in their variable domains that are often specific for different epitopes. The modifier “monoclonal” indicates the character of the antibody as being obtained from a substantially homogeneous population of antibodies and is not to be construed as requiring production of the antibody by any particular method. For example, the monoclonal antibodies to be used in accordance with the present invention may be made by the hybridoma method first described by Kohler et al., Nature 256:495 (1975) or may be made by recombinant DNA methods (see, e.g., U.S. Pat. No. 4,816,567). The “monoclonal antibodies” may also be isolated from phage antibody libraries using the techniques described in Clackson et al., Nature 352:624-628 (1991), and Marks et al., J. Mol. Biol. 222:581-597 (1991), for example. See also Presta, J. Allergy Clin. Immunol. 116:731 (2005).

As used herein, the term “gene” is used broadly to refer to any segment of nucleic acid associated with a biological function. Thus, genes include coding sequences and/or the regulatory sequences required for their expression. For example, “gene” refers to a nucleic acid fragment that expresses mRNA, functional RNA, or specific protein, including regulatory sequences. “Genes” also include nonexpressed DNA segments that, for example, form recognition sequences for other proteins. “Genes” can be obtained from a variety of sources, including cloning from a source of interest or synthesizing from known or predicted sequence information, and may include sequences designed to have desired parameters. Genes include both naturally occurring nucleotide sequences encoding a molecule of interest and synthetically derived nucleotide sequences encoding a molecule of interest, for example, complementary DNA (cDNA) obtained from a messenger RNA (mRNA) nucleotide sequence.

As used herein, the term “germline” or “germline sequence” refers to a sequence of unrearranged immunoglobulin DNA sequences. Any suitable source of unrearranged immunoglobulin sequences may be used. Human germline sequences may be obtained, for example, from JOINSOLVER® germline databases on the website for the National Institute of Arthritis and Musculoskeletal and Skin Diseases of the United States National Institutes of Health. Mouse germline sequences may be obtained, for example, as described in Giudicelli et al., Nucleic Acids Res. 33: D256-D261 (2005).

As used herein, the term “library” as used herein is, typically, a collection of related but diverse polynucleotides that are, in general, in a common vector backbone. For example, a light chain or heavy chain immunoglobulin library may contain polynucleotides, in a common vector backbone, that encode light and/or heavy chain immunoglobulins, which are diverse but related in their nucleotide sequence; for example, which immunoglobulins are functionally diverse in their abilities to form complexes with other immunoglobulins, e.g., in an antibody display system of the present invention, and bind a particular antigen.

As used herein, the term “polynucleotides” discussed herein form part of the present invention. A “polynucleotide”, “polynucleic acid”, “nucleic acid” or “nucleic acid molecule” include DNA and RNA, single- or double-stranded. Polynucleotides e.g., encoding an immunoglobulin chain or component of the antibody display system of the present invention, may, in an embodiment of the invention, be flanked by natural regulatory (expression control) sequences, or may be associated with heterologous sequences, including promoters, internal ribosome entry sites (IRES) and other ribosome binding site sequences, enhancers, response elements, suppressors, signal sequences, polyadenylation sequences, introns, 5′- and 3′-non-coding regions, and the like.

Polynucleotides e.g., encoding an immunoglobulin chain or component of the antibody display system of the present invention, may be operably associated with a promoter. A “promoter” or “promoter sequence” is, in an embodiment of the invention, a DNA regulatory region capable of binding an RNA polymerase in a cell (e.g., directly or through other promoter-bound proteins or substances) and initiating transcription of a coding sequence. A promoter sequence is, in general, bounded at its 3′ terminus by the transcription initiation site and extends upstream (5′ direction) to include the minimum number of bases or elements necessary to initiate transcription at any level. Within the promoter sequence may be found a transcription initiation site (conveniently defined, for example, by mapping with nuclease S1), as well as protein binding domains (consensus sequences) responsible for the binding of RNA polymerase. The promoter may be operably associated with other expression control sequences, including enhancer and repressor sequences or with a nucleic acid of the invention. Promoters which may be used to control gene expression include, but are not limited to, cytomegalovirus (CMV) promoter (U.S. Pat. Nos. 5,385,839 and 5,168,062), the SV40 early promoter region (Benoist, et al., Nature 290:304-310 (1981)), the promoter contained in the 3′ long terminal repeat of Rous sarcoma virus (Yamamoto et al., Cell 22:787-797 (1980)), the herpes thymidine kinase promoter (Wagner et al., Proc. Natl. Acad. Sci. USA 78:1441-1445 (1981)), the regulatory sequences of the metallothionein gene (Brinster et al., Nature 296:39-42 (1982)); prokaryotic expression vectors such as the β-lactamase promoter (Villa-Komaroff et al., Proc. Natl. Acad. Sci. USA 75:3727-3731 (1978)), or the tac promoter (DeBoer et al., Proc. Natl. Acad. Sci. USA 80:21-25 (1983)); see also “Useful proteins from recombinant bacteria” in Scientific American 242:74-94 (1980); and promoter elements from yeast or other fungi such as the Gal 4 promoter, the ADC (alcohol dehydrogenase) promoter, PGK (phosphoglycerol kinase) promoter or the alkaline phosphatase promoter.

As used herein, the terms “vector”, “cloning vector” and “expression vector” include a vehicle (e.g., a plasmid) by which a DNA or RNA sequence can be introduced into a host cell so as to transform the host and, optionally, promote expression and/or replication of the introduced sequence. Polynucleotides encoding an immunoglobulin chain or component of the antibody display system of the present invention may, in an embodiment of the invention, be in a vector.

As used herein, the terms “cell,” “cell line,” and “cell culture” are used interchangeably and all such designations include progeny. Thus, the words “transformants” and “transformed cells” include the primary subject cell and cultures derived therefrom without regard for the number of transfers. It is also understood that not all progeny will have precisely identical DNA content, due to deliberate or inadvertent mutations. Mutant progeny that have the same function or biological activity as screened for in the originally transformed cell are included. Where distinct designations are intended, it will be clear from the context.

As used herein, the term “control sequences” or “regulatory sequences” refers to DNA sequences necessary for the expression of an operably linked coding sequence in a particular host organism. The control sequences that are suitable for expression in eukaryotes, for example, include a promoter, operator or enhancer sequences, transcription termination sequences, and polyadenylation sequences for expression of a messenger RNA encoding a protein and a ribosome binding site for facilitating translation of the messenger RNA.

As used herein, a nucleic acid is “operably linked” when it is placed into a functional relationship with another nucleic acid sequence, e.g., a regulatory sequence. For example, DNA for a pre-sequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

As used herein, the term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

As used herein, the term “expression” as used herein is defined as the transcription and/or translation of a particular nucleotide sequence.

As used herein, the term “treat” or “treating” means to administer a therapeutic agent, such as a composition containing any of the human MSLN binders of the present invention, topically, subcutaneously, intramuscular, intradermally, or systemically to an individual in need. The amount of a therapeutic agent that is effective to treat cancer or proliferative disease in the individual may vary according to factors such as the injury or disease state, age, and/or weight of the individual, and the ability of the therapeutic agent to elicit a desired response in the individual. Whether the therapeutic objective has been achieved can be assessed by the individual and/or any clinical measurement typically used by physicians or other skilled healthcare providers to assess the severity or progression status of the treatment. Thus, the terms denote that a beneficial result has been or will be conferred on a human or animal individual in need.

As used herein, the term “treatment,” as it applies to a human or veterinary individual, refers to therapeutic treatment, as well as diagnostic applications. “Treatment” as it applies to a human or veterinary individual, encompasses contact of the antibodies or antigen binding fragments of the present invention to a human or animal subject.

As used herein, the term “therapeutically effective amount” refers to a quantity of a specific substance sufficient to achieve a desired effect in an individual being treated. For instance, this may be the amount necessary to inhibit or reduce the severity of a disease or disorder in an individual.

As used herein, the term “combination therapy” refers to treatment of a human or animal individual comprising administering a first therapeutic agent and a second therapeutic agent consecutively or concurrently to the individual. In general, the first and second therapeutic agents are administered to the individual separately and not as a mixture; however, there may be embodiments where the first and second therapeutic agents are mixed prior to administration.

The terms “host cell,” “host cell line,” and “host cell culture” are used interchangeably and refer to cells into which exogenous nucleic acid has been introduced, including the progeny of such cells. Host cells include “engineered cells,” “transformants,” and “transformed cells,” which include the primary engineered (e.g., transformed) cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein. As appropriate, the host cells can be stably or transiently transfected with a polynucleotide encoding a fusion protein, as described herein.

### Human MSLN Binders

The human MSLN binders of the present invention are chimeric or fully human antibodies or antigen binding fragments thereof that specifically bind human MSLN. The human MSLN binders comprise a V_{H} domain and a V_{L} domain, each domain comprising three CDRs and four Frameworks (FR) in the following arrangement

### FR1-CDR1-FR2-CDR2-FR3-CDR3-FR4.

These human MSLN binders comprise six complementarity determining regions (CDRs) comprising a particular combination of three CDRs from a V_{H} and three CDRs from the V_{L} that pairs with the V_{H}. The CDR sequences may be defined according to any numbering scheme useful for defining CDR sequences including but not limited to the Kabat, Chothia, AbM, ImMunoGeneTics (IMGT), or Contact numbering scheme. Guidance for defining the CDR sequences may be found in the general rules disclosed in www.bioinf.org.uk: Prof. Andrew C. R. Martin's Group and reproduced in Table 1.

In a particular embodiment, the CDRs are defined by Kabat or IMGT. The CDR amino acid sequences shown in Tables 2-4 are set forth according to the Kabat numbering scheme for identifying CDR amino acid sequences.

TABLE 2
Amino Acid Sequences of human MSLN binders SV017.47D7.1C2
(V_{H} and V_{L} CDRs as defined by Kabat)^{±}

| | CDR1 | CDR2 | CDR3 |
|---|---|---|---|
| VH | SYWMH | RINSDGTSTVYADSVKG | GRGIMTY |
| | (SEQ ID NO: 4) | (SEQ ID NO: 5) | (SEQ ID NO: 6) |
| VL | RASQDIGSWLT | AASSLQS | QQYKSYPLT |
| | (SEQ ID NO: 7) | (SEQ ID NO: 8) | (SEQ ID NO: 9) |

^{±}human MSLN binders SV017.13B12.1E1, SV017.50G3.1B1, and SV017.51F4.1A1 comprise CDRs having the same amino acid sequences as the CDRs of SV017.47D7.1C2

TABLE 3
Amino Acid Sequences of human MSLN binder SV018.20B12.1D1
(V_{H} and V_{L} CDRs as defined by Kabat)

| | CDR1 | CDR2 | CDR3 |
|---|---|---|---|
| VH | SYYWS | YIYYSGSTNYNPSLKS | TGYIPMDV |
| | (SEQ ID NO: 12) | (SEQ ID NO: 13) | (SEQ ID NO: 14) |
| VL | RASQSIGSNLH | YVSQSFS | HQSRSLPYT |
| | (SEQ ID NO: 15) | (SEQ ID NO: 16) | (SEQ ID NO: 17) |

TABLE 4
Amino Acid Sequences of human MSLN binder SV018.46B6.1G1
(V_{H} and V_{L} CDRs as defined by Kabat)

| | CDR1 | CDR2 | CDR3 |
|---|---|---|---|
| VH | TYGIS | WITAYNRNTNYAQKLQG | VGDSDAFDI |
| | (SEQ ID NO: 20) | (SEQ ID NO: 21) | (SEQ ID NO: 22) |
| VL | RTSQSVENSFLS | GTSTRAT | QQDYKLPLT |
| | (SEQ ID NO: 23) | (SEQ ID NO: 24) | (SEQ ID NO: 25) |

A particular CDR amino acid sequence determined using any one of the schemes for identifying CDR amino acid sequences (See Table 1) have more or less amino acids than that of CDR amino acid sequences identified according to any other numbering scheme but the CDR amino acid sequences will overlap to some extent. Thus, the CDR amino acid sequences defined according to Kabat are not to be construed as limiting and any human MSLN binder in which the CDR amino acid sequences have been identified by another numbering scheme will fall within the scope of the human MSLN binders of the present invention provided the amino acid sequences for such human MSLN binders comprise the six CDR amino acid sequences as identified by Kabat. For all human MSLN binders disclosed herein unless indicated otherwise, the amino acids comprising the variable domains as a whole are numbered according to the Kabat numbering scheme independently of how the amino acids comprising the CDR are defined. The heavy chain constant domains are numbered according to the Eu numbering scheme.

In some embodiments, provided herein is a human mesothelin binder comprises the six complementarity determining regions (CDRs) of an antibody comprising a heavy chain variable domain (V_{H}) comprising the amino acid sequence set forth in SEQ ID NO: 18 and a light chain variable domain (V_{L}) comprising the amino acid sequence set forth in SEQ ID NO: 19. The CDRs can be defined according any numbering scheme know in the art. In some embodiments, the CDRs are defined using the Kabat, Chothia, AbM, ImMunoGeneTics (IMGT), or Contact numbering scheme, or a combination thereof. In some embodiments, the CDRs are defined according a numbering scheme in Table 1.

In some embodiments, provided herein is a human mesothelin binder comprises the six CDRs of an antibody comprising a V_{H} comprising the amino acid sequence set forth in SEQ ID NO: 10 and a V_{L} comprising the amino acid sequence set forth in SEQ ID NO: 11. The CDRs can be defined according any numbering scheme know in the art. In some embodiments, the CDRs are defined using the Kabat, Chothia, AbM, ImMunoGeneTics (IMGT), or Contact numbering scheme, or a combination thereof. In some embodiments, the CDRs are defined according a numbering scheme in Table 1.

In some embodiments, provided herein is a human mesothelin binder comprises the six CDRs of an antibody comprising a V_{H} comprising the amino acid sequence set forth in SEQ ID NO: 2, 26, 27, or 28 and a V_{L} comprising the amino acid sequence set forth in SEQ ID NO: 3. The CDRs can be defined according any numbering scheme know in the art. In some embodiments, the CDRs are defined using the Kabat, Chothia, AbM, ImMunoGeneTics (IMGT), or Contact numbering scheme, or a combination thereof. In some embodiments, the CDRs are defined according a numbering scheme in Table 1.

In particular embodiments of the invention, the human MSLN binder comprises (a) a V_{H} domain comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6; and (b) a V_{L} domain comprising a CDR1 comprising the amino acid sequence set forth in SEQ ID NO: 7, a CDR2 comprising the amino acid sequence set forth in SEQ ID NO: 8, and a CDR3 comprising the amino acid sequence set forth in SEQ ID NO: 9, wherein the CDR sequences are defined by the Kabat numbering scheme.

In particular embodiments of the invention, the human MSLN binder comprises (a) a V_{H} domain comprising a CDR1 comprising the amino acid sequence set forth in SEQ ID NO: 12, a CDR2 comprising the amino acid sequence set forth in SEQ ID NO: 13, and a CDR3 comprising the amino acid sequence set forth in SEQ ID NO: 14; and (b) a V_{L} domain comprising a CDR1 comprising the amino acid sequence set forth in SEQ ID NO: 15, a CDR2 comprising the amino acid sequence set forth in SEQ ID NO: 16, and a CDR3 comprising the amino acid sequence set forth in SEQ ID NO: 17, wherein the CDR sequences are defined by the Kabat numbering scheme.

In particular embodiments of the invention, the human MSLN binder comprises (a) a V_{H} domain comprising a CDR1 comprising the amino acid sequence set forth in SEQ ID NO: 20, a CDR2 comprising the amino acid sequence set forth in SEQ ID NO: 21, and a CDR3 comprising the amino acid sequence set forth in SEQ ID NO: 22; and (b) a V_{L} domain comprising a CDR1 comprising the amino acid sequence set forth in SEQ ID NO: 23, a CDR2 comprising the amino acid sequence set forth in SEQ ID NO: 24, and a CDR3 comprising the amino acid sequence set forth in SEQ ID NO: 25, wherein the CDR sequences are defined by the Kabat numbering scheme.

In further embodiments, the mesothelin binder comprises a V_{H} domain comprising the amino acid sequence set forth in SEQ ID NO: 2, SEQ ID NO: 26, SEQ ID NO: 27, or SEQ ID NO: 28 and a V_{L} domain comprising the amino acid sequence set forth in SEQ ID NO: 3.

In some embodiments, the mesothelin binder comprises a V_{H} comprising the amino acid sequence set forth in SEQ ID NO: 2; and a V_{L} comprising the amino acid sequence set forth in SEQ ID NO: 3.

In some embodiments, the mesothelin binder comprises a V_{H} comprising an amino acid sequence having the 2nd amino acid residue to the 116th amino acid residue of the amino acid sequence ranging from the 2nd amino acid residue to the 116th amino acid residue set forth in SEQ ID NO: 2 and the 1st amino acid residue of the amino acid sequence being pyroglutamate; and a V_{L} comprising the amino acid sequence set forth in SEQ ID NO: 3.

In some embodiments, the mesothelin binder comprises a V_{H} comprising the amino acid sequence set forth in SEQ ID NO: 26; and a V_{L} comprising the amino acid sequence set forth in SEQ ID NO: 3.

In some embodiments, the mesothelin binder comprises a V_{H} comprising an amino acid sequence having the 2nd amino acid residue to the 116th amino acid residue of the amino acid sequence ranging from the 2nd amino acid residue to the 116th amino acid residue set forth in SEQ ID NO: 26 and the 1st amino acid residue of the amino acid sequence being pyroglutamate; and a V_{L} comprising the amino acid sequence set forth in SEQ ID NO: 3.

In some embodiments, the mesothelin binder comprises a V_{H} comprising the amino acid sequence set forth in SEQ ID NO: 27; and a V_{L} comprising the amino acid sequence set forth in SEQ ID NO: 3.

In some embodiments, the mesothelin binder comprises a V_{H} comprising an amino acid sequence having the 2nd amino acid residue to the 116th amino acid residue of the amino acid sequence ranging from the 2nd amino acid residue to the 116th amino acid residue set forth in SEQ ID NO: 27 and the 1st amino acid residue of the amino acid sequence being pyroglutamate; and a V_{L} comprising the amino acid sequence set forth in SEQ ID NO: 3.

In some embodiments, the mesothelin binder comprises a V_{H} comprising the amino acid sequence set forth in SEQ ID NO: 28; and a V_{L} comprising the amino acid sequence set forth in SEQ ID NO: 3.

In some embodiments, the mesothelin binder comprises a V_{H} comprising an amino acid sequence having the 2nd amino acid residue to the 116th amino acid residue of the amino acid sequence ranging from the 2nd amino acid residue to the 116th amino acid residue set forth in SEQ ID NO: 28 and the 1st amino acid residue of the amino acid sequence being pyroglutamate; and a V_{L} comprising the amino acid sequence set forth in SEQ ID NO: 3.

In further embodiments, the mesothelin binder comprises a V_{H} domain comprising the amino acid sequence set forth in SEQ ID NO: 10 and a V_{L} domain comprising the amino acid sequence set forth in SEQ ID NO: 11.

In further embodiments, the mesothelin binder comprises a V_{H} comprising the amino acid sequence set forth in SEQ ID NO: 10; and a V_{L} comprising the amino acid sequence set forth in SEQ ID NO: 11.

In further embodiments, the mesothelin binder comprises a V_{H} comprising an amino acid sequence having the 2nd amino acid residue to the 116th amino acid residue of the amino acid sequence ranging from the 2nd amino acid residue to the 116th amino acid residue set forth in SEQ ID NO: 10 and the 1st amino acid residue of the amino acid sequence being pyroglutamate; and a V_{L} comprising the amino acid sequence set forth in SEQ ID NO: 11.

In further embodiments, the mesothelin binder comprises a V_{H} comprising the amino acid sequence set forth in SEQ ID NO: 10; and a V_{L} comprising an amino acid sequence having the 2nd amino acid residue to the 107th amino acid residue of the amino acid sequence ranging from the 2nd amino acid residue to the 107th amino acid residue set forth in SEQ ID NO: 11 and the 1st amino acid residue of the amino acid sequence being pyroglutamate.

In further embodiments, the mesothelin binder comprises a V_{H} comprising an amino acid sequence having the 2nd amino acid residue to the 116th amino acid residue of the amino acid sequence ranging from the 2nd amino acid residue to the 116th amino acid residue set forth in SEQ ID NO: 10 and the 1st amino acid residue of the amino acid sequence being pyroglutamate; and a V_{L} comprising an amino acid sequence having the 2nd amino acid residue to the 107th amino acid residue of the amino acid sequence ranging from the 2nd amino acid residue to the 107th amino acid residue set forth in SEQ ID NO: 11 and the 1st amino acid residue of the amino acid sequence being pyroglutamate.

In further embodiments, the mesothelin binder comprises a V_{H} comprising the amino acid sequence set forth in SEQ ID NO: 18; and a V_{L} comprising the amino acid sequence set forth in SEQ ID NO: 19.

In further embodiments, the mesothelin binder comprises a V_{H} comprising an amino acid sequence having the 2nd amino acid residue to the 118th amino acid residue of the amino acid sequence ranging from the 2nd amino acid residue to the 118th amino acid residue set forth in SEQ ID NO: 18 and the 1st amino acid residue of the amino acid sequence being pyroglutamate; and a V_{L} comprising the amino acid sequence set forth in SEQ ID NO: 19.

In further embodiments, the mesothelin binder comprises a V_{H} comprising the amino acid sequence set forth in SEQ ID NO: 18; and a V_{L} comprising an amino acid sequence having the 2nd amino acid residue to the 108th amino acid residue of the amino acid sequence ranging from the 2nd amino acid residue to the 108th amino acid residue set forth in SEQ ID NO: 19 and the 1st amino acid residue of the amino acid sequence being pyroglutamate.

In further embodiments, the mesothelin binder comprises a V_{H} comprising an amino acid sequence having the 2nd amino acid residue to the 118th amino acid residue of the amino acid sequence ranging from the 2nd amino acid residue to the 118th amino acid residue set forth in SEQ ID NO: 18 and the 1st amino acid residue of the amino acid sequence being pyroglutamate; and a V_{L} comprising an amino acid sequence having the 2nd amino acid residue to the 108th amino acid residue of the amino acid sequence ranging from the 2nd amino acid residue to the 108th amino acid residue set forth in SEQ ID NO: 19 and the 1st amino acid residue of the amino acid sequence being pyroglutamate.

In further embodiments of the invention, the human MSLN binder is an antibody comprising a heavy chain (HC) constant domain of the IgG1 isotype. In particular embodiments, the heavy chain constant domain comprises 1, 2, 3, 4, 5, 6, 7, 8, 9, or 10 amino acid substitutions, additions, deletions, or combinations thereof compared to the amino acid sequence of the native IgG1 isotype.

IgG1 heavy chain constant domain comprising the amino acid sequence shown in SEQ ID NO: 17 or a variant thereof comprising 1, 2, 3, 4, 5, 6, 7, 8, 9, or 10 amino acid substitutions, additions, deletions, or combinations thereof.

In particular embodiments of the invention, the constant domains as disclosed herein may comprise a C-terminal lysine or lack either a C-terminal lysine or a C-terminal glycine-lysine dipeptide.

In any one of the embodiments disclosed herein, the light chain may comprise a human kappa light chain constant domain comprising SEQ ID NO: 38.

### Human MSLN Binders Comprising an Effector-Silent Fc Domain

Effector-silent human MSLN binders of the present invention that comprise full-sized antibodies may comprise an HC constant domain or Fc domain thereof that has been modified such that the antibody displays no measurable binding to one or more FcRs or displays reduced binding to one or more FcRs compared to that of an unmodified antibody of the same IgG isotype. The effector-silent antibodies may in further embodiments display no measurable binding to each of FcγRIIIa, FcγRIIa, and FcγRI or display reduced binding to each of FcγRIIIa, FcγRIIa, and FcγRI compared to that of an unmodified antibody of the same IgG isotype. In particular embodiments, the HC constant domain or Fc domain is a human HC constant domain or Fc domain.

In particular embodiments, the effector-silent antibody comprises an Fc domain of an IgG1 isotype that has been modified to lack N-glycosylation of the asparagine (Asn) residue at position 297 (Eu numbering system) of the HC constant domain. The consensus sequence for N-glycosylation is Asn-Xaa-Ser/Thr (wherein Xaa at position 298 is any amino acid except Pro); the N-glycosylation consensus sequence is Asn-Ser-Thr. The modification may be achieved by replacing the codon encoding the Asn at position 297 in the nucleic acid molecule encoding the HC constant domain with a codon encoding another amino acid, for example Ala, Asp, Gln, Gly, or Glu, e.g., N297A, N297Q, N297G, N297E, or N297D. Alternatively, the codon for Ser at position 298 may be replaced with the codon for Pro or the codon for Thr at position 299 may be replaced with any codon except the codon for Ser. In a further alternative each of the amino acids comprising the N-glycosylation consensus sequence is replaced with another amino acid. Such modified IgG molecules have no measurable effector function. In particular embodiments, these mutated HC molecules may further comprise 1, 2, 3, 4, 5, 6, 7, 8, 9, or 10 additional amino acid substitutions, insertions, and/or deletions, wherein said substitutions may be conservative mutations or non-conservative mutations. In further embodiments, such IgGs modified to lack N-glycosylation at position 297 may further include one or more additional mutations disclosed herein for eliminating measurable effector function.

An exemplary IgG1 HC constant domain mutated at position 297, which abolishes the N-glycosylation of the HC constant domain, is set forth in SEQ ID NO: 36. In particular embodiments, these mutated HC molecules may further comprise 1, 2, 3, 4, 5, 6, 7, 8, 9, or 10 additional amino acid substitutions, insertions, and/or deletions, wherein said substitutions may be conservative mutations or non-conservative mutations.

In particular embodiments, the Fc domain of the IgG1 HC constant domain comprising the effector-silent antibody is modified to include one or more amino acid substitutions selected from E233P, L234A, L235A, L235E, N297A, N297D, D265S, and P331S (wherein the positions are identified according to Eu numbering) and wherein said HC constant domain is effector-silent. In particular embodiments, the modified IgG1 further comprise 1, 2, 3, 4, 5, 6, 7, 8, 9, or 10 additional amino acid substitutions, insertions, and/or deletions, wherein said substitutions may be conservative mutations or non-conservative mutations.

In particular embodiments, the HC constant domain comprises L234A, L235A, and D265S substitutions (wherein the positions are identified according to Eu numbering). In particular embodiments, the HC constant domain comprises an amino acid substitution at position Pro329 and at least one further amino acid substitution selected from E233P, L234A, L235A, L235E, N297A, N297D, D265S, and P331S (wherein the positions are identified according to Eu numbering). These and other substitutions are disclosed in WO9428027; WO2004099249; WO20121300831, U.S. Pat. Nos. 9,708,406; 8,969,526; 9,296,815; Sondermann et al. Nature 406, 267-273 (2000)).

In particular embodiments of the above, the HC constant domain comprises an L234A/L235A/D265A; L234A/L235A/P329G; L235E; D265A; D265A/N297G; or V234A/G237A/P238S/H268A/V309L/A330S/P331S substitutions, wherein the positions are identified according to Eu numbering. In particular embodiments, the HC molecules further comprise 1, 2, 3, 4, 5, 6, 7, 8, 9, or 10 additional amino acid substitutions, insertions, and/or deletions, wherein said substitutions may be conservative mutations or non-conservative mutations.

In particular embodiments, the effector-silent antibody comprises an IgG1 isotype, in which the Fc domain of the HC constant domain has been modified to be effector-silent by substituting the amino acids from position 233 to position 236 of the IgG1 with the corresponding amino acids of the human IgG2 HC and substituting the amino acids at positions 327, 330, and 331 with the corresponding amino acids of the human IgG4 HC, wherein the positions are identified according to Eu numbering (Armour et al., Eur. J. Immunol. 29 (8): 2613-24 (1999); Shields et al., J. Biol. Chem. 276 (9): 6591-604 (2001)). In particular embodiments, the modified IgG1 further comprise 1, 2, 3, 4, 5, 6, 7, 8, 9, or 10 additional amino acid substitutions, insertions, and/or deletions, wherein said substitutions may be conservative mutations or non-conservative mutations.

In particular embodiments, the effector-silent antibody comprises a VH fused or linked to a hybrid human immunoglobulin HC constant domain, which includes a hinge region, a CH2 domain and a CH3 domain in an N-terminal to C-terminal direction, wherein the hinge region comprises an at least partial amino acid sequence of a human IgD hinge region or a human IgG1 hinge region; and the CH2 domain is of a human IgG4 CH2 domain, a portion of which, at its N-terminal region, is replaced by 4-37 amino acid residues of an N-terminal region of a human IgG2 CH2 or human IgD CH2 domain. Such hybrid human HC constant domain is disclosed in U.S. Pat. No. 7,867,491, which is incorporated herein by reference in its entirety.

Exemplary IgG1 HC constant domains include HC constant domains comprising an amino acid sequence selected from the group consisting of amino acid sequences set forth in SEQ ID NO: 30, SEQ ID NO: 31, SEQ ID NO: 32, SEQ ID NO: 33, SEQ ID NO: 34, SEQ ID NO: 35, SEQ ID NO: 36, and SEQ ID NO: 37.

In particular embodiments of the human MSLN binder, the human MSLN binder is an antibody comprising an IgG1 Fc domain as disclosed herein, which further comprises a C-terminal lysine or lack either a C-terminal lysine or a C-terminal glycine-lysine dipeptide.

In any one of the embodiments disclosed herein, the light chain may comprise a human kappa light chain constant domain comprising SEQ ID NO: 38.

Alternatively, or additionally, in another embodiment of the present disclosure, the human MSLN binders and other peptides provided herein may undergo post-translational modifications as known in the art. Examples of post-translational modifications include, but are not limited to, chemical modifications, such as disulfide bonds, oligosaccharides, N-terminal pyroglutamate formation, C-terminal lysine processing (such that lysine is removed), deamidation, isomerization, oxidation, glycation, peptide bond cleavage, non-reductible cross-linking, truncation and others known in the art. See, Liu, et. al., Heterogeneity of Monoclonal Antibodies, J. Pharma. Sci. vol. 97, no. 7, pp. 2426-2447 (July 2008). Other types of modifications include noncovalent interaction, conformational heterogeneity, and aggregation. Id.

In some embodiments, an N-terminal E or Q of a human MSLN binder provided herein is substituted with pyroglutamate. In some embodiments, a C-terminal K of a human MSLN binder provided herein is removed. In other embodiments, an N-terminal E or Q of a human MSLN binder provided herein is substituted with pyroglutamate and a C-terminal K of the human MSLN binder (e.g., heavy chain C terminal amino acid) is removed. The present disclosure includes any of the above described post-translational modifications of any of the human MSLN binders and polypeptides provided herein. For example, in some embodiment, provided herein is a human MSLN binder that comprises the same sequences as 47D7, except that the first N-terminal amino acid of the VH region is substituted with pyroglutamate; and/or the C-terminal amino acid of the heavy chain is removed. In some embodiment, provided herein is a human MSLN binder that comprises the same sequences as 20B12, except that the first N-terminal amino acid of the VH region is substituted with pyroglutamate; the first N-terminal amino acid of the VL region is substituted with pyroglutamate; and/or the C-terminal amino acid of the heavy chain is removed. In some embodiment, provided herein is a human MSLN binder that comprises the same sequences as 45B6, except that the first N-terminal amino acid of the VH region is substituted with pyroglutamate; the first N-terminal amino acid of the VL region is substituted with pyroglutamate; and/or the C-terminal amino acid of the heavy chain is removed.

In some specific embodiments, provided herein is an antibody or fragment thereof (e.g., scFv) or a polyprotein comprising an amino acid sequence set forth in any one of SEQ ID NOs: 2, 10, 11, 18, 19, and 26 to 28, except that the first amino acid at the N-terminus is substituted with pyroglutamate.

In other specific embodiments, provided herein is antibody or fragment thereof comprising an amino acid sequence set forth in any one of SEQ ID NOs: 29-37, except that the C-terminal K is removed.

ScFv Fusion Proteins that Bind Human MSLN

In particular embodiments, the V_{H} and V_{L} disclosed herein are expressed as an ScFv fusion protein in which the V_{L} and V_{H} domains are linked together by a peptide linker. The peptide linker joins the carboxyl terminus of one variable region domain to the amino terminus of the other variable domain without compromising the fidelity of the V_{H}-V_{L} paring and antigen-binding sites. Thus, the ScFv may comprise a fusion protein in which the C-terminus of a V_{L} is linked by a peptide linker to the N-terminus of a V_{H} or a fusion protein in which the C-terminus of a V_{H} is linked by a peptide linker to the N-terminus of a V_{L}. Peptide linkers for linking the variable domains can vary from 10 to 25 amino acids in length and are typically, but not always, composed of hydrophilic amino acids such as glycine (G) and serine(S) having the structure G4S, for example, (G4S), wherein n is 1, 2, 3, 4, or 5. Peptide linkers of shorter lengths (0-4 amino acids) have also been used; however, ScFv bearing shorter linkers may form multimers. Generally, the (G4S) 3 peptide comprising three repeating G4S units is used as an ScFv peptide linker (See for example, Leath et al., Int. J. Oncol. 24:765-771 (2004); Holliger et al., Proc. Natl. Acad. Sci. USA 90:6444-6448 (1993); Iliades et al., FEBS Lett. 409:437-441 (1997)).

Exemplary ScFv fusion proteins include the structure V_{L}-(G4S)ₙ-V_{H} or V_{H}-(G4S)ₙ-V_{L} wherein (a) V_{L} comprises the amino acid sequence set forth in SEQ ID NO: 3 and V_{H} comprises the amino acid sequence set forth in SEQ ID NO: 2; (b) V_{L} comprises the amino acid sequence set forth in SEQ ID NO: 3 and V_{H} comprises the amino acid sequence set for in SEQ ID NO: 26; (c) V_{L} comprises the amino acid sequence set forth in SEQ ID NO: 3 and V_{H} comprises the amino acid sequence set for in SEQ ID NO: 27; (d) V_{L} comprises the amino acid sequence set forth in SEQ ID NO: 3 and V_{H} comprises the amino acid sequence set for in SEQ ID NO: 28; (e) V_{L} comprises the amino acid sequence set forth in SEQ ID NO: 11 and V_{H} comprises the amino acid sequence set for in SEQ ID NO: 10; or (f) V_{L} comprises the amino acid sequence set forth in SEQ ID NO: 19 and V_{H} comprises the amino acid sequence set for in SEQ ID NO: 18, wherein n is 1, 2, 3, 4, or 5.

The ScFvs disclosed herein may be provided in a bispecific format comprising a CD3 binder (ScFv) linked by a peptide linker to an ScFv that binds HSLN as disclosed herein. When these molecules, called Bispecific T-cell engagers (BiTE®s), bind CD3 on T cells and HSLN expressed on the surface of a cell, it brings the T cells to a tumor site.

ScFvs disclosed herein may also be fused to cellular toxins, radioisotopes, cytokines, and enzymes for cancer, autoimmune, and/or inflammatory therapeutic applications. In particular embodiments, the peptide linker may comprise one to 10 G4S peptide units.

In further embodiments, the ScFvs disclosed herein may be linked to or inserted in different locations of an intact IgG molecule to confer dual epitope binding. For example, a bispecific antibody may be provided comprising two heterodimeric heavy chain constant domains wherein the N-terminus of one heavy chain constant domain is fused to the C-terminus of an ScFv disclosed herein and the N-terminus of the other heavy chain constant domain is fused to the C-terminus of an ScFv that targets an antigen other than human MSLN or a Fab′ that targets an antigen other than human MSLN.

### Nucleic Acid Molecules Encoding the Human MSLN Binders

The present disclosure further provides nucleic acid molecules that encode the human MSLN binders of the present disclosure. In particular embodiments, the human MSLN binder comprises a V_{H} domain encoded by a nucleic acid molecule and a V_{L} encoded by a nucleic acid molecule. Nucleic acid sequences encoding the HSLN binders disclosed herein may be obtained by back-translating the amino acid sequence of the HSLN binder into a nucleic acid sequence that encodes the HSLN binder. The codons of the nucleic acid molecule so obtained may be further modified to correspond to codons commonly or more efficiently used when translated in a particular cell type. Methods and computer programs for back-translating and/or optimizing a nucleic acid molecule for enhancing expression in a particular host cell are well known in the art, e.g., the IDT Codon Optimization Tool commercially available from Integrated DNA Technologies, Inc. 1710 Commercial Park, Coralville, Iowa 52241, USA.; U.S. Pat. No. 8,326,547; WO2020024917A1.

In particular embodiments, the HC and LC (or V_{H} and V_{L}) are expressed as a fusion protein in which the N-terminus of the HC and the LC (or V_{H} and V_{L}) are fused at the N-terminus to a leader peptide to facilitate the transport of the antibody through the secretory pathway. In particular embodiments, the N-terminus of the ScFv fusion protein is fused at the N-terminus to a leader or signal peptide to facilitate the transport of the antibody through the secretory pathway. Examples of leader/signal peptides that may be used those comprising the amino acid sequence set forth in SEQ ID NO: 39 or SEQ ID NO: 40. Thus, in particular embodiments, the aforementioned nucleic acid molecules may comprise a polynucleotide encoding a leader peptide linked to the 5′ end of the nucleic acid molecule encoding the HSLN binder.

The nucleic acid molecules disclosed herein may include one or more substitutions that optimize one or more of the codons for enhancing the expression of the nucleic acid molecule in a particular host cell, e.g., yeast or fungal host cell, non-human mammalian host cell, human host cell, insect host cell, or prokaryote host cell.

### Methods For Making Human MSLN Binders

The present disclosure includes recombinant methods for making human MSLN binders comprising introducing into a host cell (i) an expression vector that encodes the V_{H} and V_{L} of a human MSLN binder or the HC and LC of a human MSLN binder, or (ii) two expression vectors, one encoding the V_{H} of a human MSLN binder or the HC of a human MSLN binder the other encoding the V_{L} of a human MSLN binder or the LC of a human MSLN binder. The nucleic acid molecules or polynucleotides encoding the V_{H}, V_{L}, HC, or LC are operably linked to a promoter and other transcription and translation regulatory sequences. The host cell is cultured under conditions and a time period suitable for expression of the nucleic acid molecules followed by isolating the mesothelin binder from the host cell and/or medium in which the host cell is grown. See e.g., WO2004041862, WO2006122786, WO2008020079, WO2008142164 or WO2009068627. The expression vector may be a plasmid or viral vector. The disclosure also relates to hosts or host cells that contain such nucleic acid molecule encoding the Arginase 1 binders or components thereof, e.g., solely the V_{H} or HC or solely the V_{L} or HC

Eukaryotic and prokaryotic host cells, including mammalian cells as hosts for expression of the human MSLN binder are well known in the art and include many immortalized cell lines available from the American Type Culture Collection (ATCC). These include, but are not limited to, Chinese hamster ovary (CHO) cells, NSO, SP2 cells, HeLa cells, baby hamster kidney (BHK) cells, monkey kidney cells (COS), human hepatocellular carcinoma cells (e.g., Hep G2), A549 cells, 3T3 cells, HEK-293 cells and a number of other cell lines. Thus, mammalian host cells include human, mouse, rat, dog, monkey, pig, goat, bovine, horse, and hamster cells. Cell lines of particular preference are selected through determining which cell lines have high expression levels. Other cell lines that may be used are insect cell lines (e.g., Spodoptera frugiperda or Trichoplusia ni), amphibian cells, bacterial cells, plant cells and fungal cells. Fungal cells include yeast and filamentous fungus cells including, for example, Pichia pastoris, Saccharomyces cerevisiea, and Trichoderma reesei. The present disclosure includes any host cell comprising a human MSLN binder of the present disclosure or comprising one or more nucleic acid molecules encoding such a human MSLN binder or comprising an expression vector that comprises one or more nucleic acid molecules encoding such human MSLN binder.

Further, expression of a human MSLN binder from production cell lines can be enhanced using a number of known techniques. For example, the glutamine synthetase gene expression system (the GS system) is a common approach for enhancing expression under certain conditions. The GS system is discussed in whole or part in connection with European Patent Nos. 0216846B1, 0256055B1, 0323997B1, and 0338841B1. Thus, in an embodiment of the disclosure, the mammalian host cells lack a glutamine synthetase gene and are grown in the absence of glutamine in the medium wherein, however, the nucleic acid molecule encoding the immunoglobulin chain comprises a glutamine synthetase gene which complements the lack of the gene in the host cell. Such host cells containing the human MSLN binder or nucleic acid(s) or expression vector(s) as discussed herein as well as expression methods, as discussed herein, for making the human MSLN binder using such a host cell are part of the present disclosure.

The present disclosure includes methods for purifying a human MSLN binder comprising introducing a sample (e.g., culture medium, cell lysate or cell lysate fraction, e.g., a soluble fraction of the lysate) comprising the human MSLN binder to a purification medium (e.g., cation-exchange medium, anion-exchange medium and/or hydrophobic exchange medium) and either collecting purified human MSLN binder from the flow-through fraction of said sample that does not bind to the medium; or, discarding the flow-through fraction and eluting bound human MSLN binder from the medium and collecting the eluate. In an embodiment of the disclosure, the medium is in a column to which the sample is applied. In an embodiment of the disclosure, the purification method is conducted following recombinant expression of the human MSLN binder in a host cell, e.g., wherein the host cell is first lysed and, optionally, the lysate is purified of insoluble materials prior to purification on a medium; or wherein the human MSLN binder is secreted into the culture medium by the host cell and the medium or a fraction thereof is applied to the purification medium.

In general, glycoproteins produced in a particular cell line or transgenic animal will have a glycosylation pattern that is characteristic for glycoproteins produced in the cell line or transgenic animal. Therefore, the particular glycosylation pattern of a human MSLN binder will depend on the particular cell line or transgenic animal used to produce the human MSLN binder. Human MSLN binders comprising only non-fucosylated N-glycans are part of the present disclosure and may be advantageous, because non-fucosylated antibodies have been shown to typically exhibit more potent efficacy than their fucosylated counterparts both in vitro and in vivo (See for example, Shinkawa et al., J. Biol. Chem. 278:3466-3473 (2003); U.S. Pat. Nos. 6,946,292 and 7,214,775). These human MSLN binders with non-fucosylated N-glycans are not likely to be immunogenic because their carbohydrate structures are a normal component of the population that exists in human serum IgG.

The present disclosure includes human MSLN binders comprising N-linked glycans that are typically added to immunoglobulins produced in Chinese hamster ovary cells (CHO N-linked glycans) or to engineered yeast cells (engineered yeast N-linked glycans), such as, for example, Pichia pastoris. For example, in an embodiment of the disclosure, the mesothelin binder comprises one or more of the “engineered yeast N-linked glycans” or “CHO N-linked glycans” (e.g., G0 and/or G0-F and/or G1 and/or G1-F and/or and/or G2-F and/or Man5). In an embodiment of the disclosure, the human MSLN binder comprises the engineered yeast N-linked glycans, i.e., G0 and/or G1 and/or G2, optionally, further including Man5. In an embodiment of the disclosure, the human MSLN binders comprise the CHO N-linked glycans, i.e., G0-F, G1-F and G2-F, optionally, further including G0 and/or G1 and/or G2 and/or Man5. In an embodiment of the disclosure, about 80% to about 95% (e.g., about 80-90%, about 85%, about 90% or about 95%) of all N-linked glycans on the human MSLN binders are engineered yeast N-linked glycans or CHO N-linked glycans. See Nett et al. Yeast. 28:237-252 (2011); Hamilton et al. Science. 313:1441-1443 (2006); Hamilton et al. Curr Opin Biotechnol. 18 (5): 387-392 (2007). For example, in an embodiment of the disclosure, an engineered yeast cell is GFI5.0 or YGLY8316 or strains set forth in U.S. Pat. No. 7,795,002 or Zha et al. Methods Mol Biol. 988:31-43 (2013). See also international patent application publication no. WO2013066765.

### Administration/Pharmaceutical Compositions

The human MSLN binder may be provided in suitable pharmaceutical compositions comprising the human MSLN binder and a pharmaceutically acceptable carrier. The carrier may be a diluent, adjuvant, excipient, or vehicle with which the human MSLN binder is administered. Such vehicles may be liquids, such as water and oils, including those of petroleum, animal, vegetable or synthetic origin, such as peanut oil, soybean oil, mineral oil, sesame oil and the like. For example, 0.4% saline and 0.3% glycine may be used. These solutions are sterile and generally free of particulate matter. They may be sterilized by conventional, well-known sterilization techniques (e.g., filtration). The compositions may contain pharmaceutically acceptable auxiliary substances as required to approximate physiological conditions such as pH adjusting and buffering agents, stabilizing, thickening, lubricating and coloring agents, etc. The concentration of the molecules or of the disclosure in such pharmaceutical formulation may vary widely, i.e., from less than about 0.5%, usually to at least about 1% to as much as 15 or 20% by weight and will be selected primarily based on required dose, fluid volumes, viscosities, etc., according to the particular mode of administration selected. Suitable vehicles and formulations, inclusive of other human proteins, e.g., human serum albumin, are described, for example, in e.g. Remington: The Science and Practice of Pharmacy, 21.sup.st Edition, Troy, D. B. ed., Lipincott Williams and Wilkins, Philadelphia, Pa. 2006, Part 5, Pharmaceutical Manufacturing pp 691-1092, see especially pp. 958-989.

The mode of administration of the human MSLN binder may be any suitable route such as parenteral administration, e.g., intradermal, intramuscular, intraperitoneal, intravenous or subcutaneous, pulmonary, transmucosal (oral, intranasal, intravaginal, rectal) or other means appreciated by the skilled artisan, as well known in the art.

The human MSLN binder may be administered to an individual (e.g., patient) by any suitable route, for example parentally by intravenous (i.v.) infusion or bolus injection, intramuscularly or subcutaneously, or intraperitoneally. i.v. infusion may be given over for, example, 15, 30, 60, 90, 120, 180, or 240 minutes, or from 1, 2, 3, 4, 5, 6, 7, 8, 9, 10, 11 or 12 hours.

The dose given to an individual having cancer or malignancy is sufficient to alleviate or at least partially arrest the disease being treated (“therapeutically effective amount”) and may be sometimes 0.005 mg/kg to about 100 mg/kg, e.g. about 0.05 mg/kg to about 30 mg/kg or about 5 mg to about 25 mg/kg, or about 4 mg/kg, about 8 mg/kg, about 16 mg/kg or about 24 mg/kg, or, e.g., about 1, 2, 3, 4, 5, 6, 7, 8, 9 or 10 mg/kg, but may even higher, for example about 15, 16, 17, 18, 19, 20, 21, 22, 23, 24, 25, 30, 40, 50, 60, 70, 80, 90 or 100 mg/kg.

A fixed unit dose may also be given, for example, 50, 100, 200, 500 or 1000 mg, or the dose may be based on the patient's surface area, e.g., 500, 400, 300, 250, 200, or 100 mg/m². Usually between 1 and 8 doses, (e.g., 1, 2, 3, 4, 5, 6, 7 or 8) may be administered to treat cancer or malignancy, but 9, 10, 11, 12, 13, 14, 15, 16, 17, 18, 19, 20 or more doses may be given.

The administration of the human MSLN binder may be repeated after one day, two days, three days, four days, five days, six days, one week, two weeks, three weeks, one month, five weeks, six weeks, seven weeks, two months, three months, four months, five months, six months or longer. Repeated courses of treatment are also possible, as is chronic administration. The repeated administration may be at the same dose or at a different dose. For example, the human MSLN binder in the methods of the disclosure may be administered at 8 mg/kg or at 16 mg/kg at weekly interval for 8 weeks, followed by administration at 8 mg/kg or at 16 mg/kg every two weeks for an additional 16 weeks, followed by administration at 8 mg/kg or at 16 mg/kg every four weeks by intravenous infusion.

The human MSLN binder may be administered by maintenance therapy, such as, e.g., once a week for a period of 6 months or more. For example, human MSLN binder in the methods of the disclosure may be provided as a daily dosage in an amount of about 0.1-100 mg/kg, such as 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day, on at least one of day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively, at least one of week 1, 2, 3, 4, 5, 6, 7, 8, 9, 10, 11, 12, 13, 14, 15, 16, 17, 18, 19 or 20 after initiation of treatment, or any combination thereof, using single or divided doses of every 24, 12, 8, 6, 4, or 2 hours, or any combination thereof.

The human MSLN binder may also be administered prophylactically in order to reduce the risk of developing cancer, delay the onset of the occurrence of an event in cancer progression, and/or reduce the risk of recurrence when a cancer is in remission. This may be especially useful in patients wherein it is difficult to locate a tumor that is known to be present due to other biological factors.

The human MSLN binder may be lyophilized for storage and reconstituted in a suitable carrier prior to use. This technique has been shown to be effective with conventional protein preparations and well known lyophilization and reconstitution techniques can be employed.

### Combination Therapy Treatments

The combination therapy of the present disclosure comprises a human MSLN binder and another therapeutic agent (small molecule or antibody) may be used for the treatment any proliferative disease, in particular, treatment of cancer. In particular embodiments, the combination therapy of the present disclosure may be used to treat melanoma, non-small cell lung cancer, head and neck cancer, urothelial cancer, breast cancer, gastrointestinal cancer, multiple myeloma, hepatocellular cancer, non-Hodgkin lymphoma, renal cancer, Hodgkin lymphoma, mesothelioma, ovarian cancer, small cell lung cancer, esophageal cancer, anal cancer, biliary tract cancer, colorectal cancer, cervical cancer, thyroid cancer, or salivary cancer.

In another embodiment, the combination therapy of the present disclosure may be used to treat pancreatic cancer, bronchus cancer, prostate cancer, pancreatic cancer, stomach cancer, ovarian cancer, urinary bladder cancer, brain or central nervous system cancer, peripheral nervous system cancer, uterine or endometrial cancer, cancer of the oral cavity or pharynx, liver cancer, kidney cancer, testicular cancer, biliary tract cancer, small bowel or appendix cancer, adrenal gland cancer, osteosarcoma, chondrosarcoma, or cancer of hematological tissues.

### Combination Therapy Comprising a Human MSLN Binder and a Chemotherapy Agent

The combination therapy of the present disclosure may be administered to an individual having a cancer in combination with chemotherapy. The individual may undergo the chemotherapy at the same time the individual is undergoing the combination therapy of the present disclosure. The individual may undergo the combination therapy of the present disclosure after the individual has completed chemotherapy. The individual may be administered the chemotherapy after completion of the combination therapy. The combination therapy of the present disclosure may also be administered to an individual having recurrent or metastatic cancer with disease progression or relapse cancer and who is undergoing chemotherapy or who has completed chemotherapy.

The chemotherapy may include a chemotherapy agent selected from the group consisting of
(i) alkylating agents, including but not limited to, bifunctional alkylators, cyclophosphamide, mechlorethamine, chlorambucil, and melphalan;
(ii) monofunctional alkylators, including but not limited to, dacarbazine, nitrosoureas, and temozolomide (oral dacarbazine);
(iii) anthracyclines, including but not limited to, daunorubicin, doxorubicin, epirubicin, idarubicin, mitoxantrone, and valrubicin;
(iv) cytoskeletal disruptors (taxanes), including but not limited to, paclitaxel, docetaxel, abraxane, and taxotere;
(v) epothilones, including but not limited to, ixabepilone, and utidelone;
(vi) histone deacetylase inhibitors, including but not limited to, vorinostat, and romidepsin;
(vii) inhibitors of topoisomerase i, including but not limited to, irinotecan, and topotecan;
(viii) inhibitors of topoisomerase ii, including but not limited to, etoposide, teniposide, and tafluposide;
(ix) kinase inhibitors, including but not limited to, bortezomib, erlotinib, gefitinib, imatinib, vemurafenib, and vismodegib;
(x) nucleotide analogs and precursor analogs, including but not limited to, azacitidine, azathioprine, fluoropyrimidines (e.g., such as capecitabine, carmofur, doxifluridine, fluorouracil, and tegafur) cytarabine, gemcitabine, hydroxyurea, mercaptopurine, methotrexate, and tioguanine (formerly thioguanine);
(xi) peptide antibiotics, including but not limited to, bleomycin and actinomycin; a platinum-based agent, including but not limited to, carboplatin, cisplatin, and oxaliplatin;
(xii) retinoids, including but not limited to, tretinoin, alitretinoin, and bexarotene; and
xiii) vinca alkaloids and derivatives, including but not limited to, vinblastine, vincristine, vindesine, and vinorelbine.

Selecting a dose of the chemotherapy agent for chemotherapy depends on several factors, including the serum or tissue turnover rate of the entity, the level of symptoms, the immunogenicity of the entity, and the accessibility of the target cells, tissue or organ in the individual being treated. The dose of the additional therapeutic agent should be an amount that provides an acceptable level of side effects. Accordingly, the dose amount and dosing frequency of each additional therapeutic agent will depend in part on the particular therapeutic agent, the severity of the cancer being treated, and patient characteristics. Guidance in selecting appropriate doses of antibodies, cytokines, and small molecules are available. See, e.g., Wawrzynczak (1996) Antibody Therapy, Bios Scientific Pub. Ltd, Oxfordshire, UK; Kresina (ed.) (1991) Monoclonal Antibodies, Cytokines and Arthritis, Marcel Dekker, New York, NY; Bach (ed.) (1993) Monoclonal Antibodies and Peptide Therapy in Autoimmune Diseases, Marcel Dekker, New York, NY; Baert et al. (2003) New Engl. J. Med. 348:601-608; Milgrom et al. (1999) New Engl. J. Med. 341:1966-1973; Slamon et al. (2001) New Engl. J. Med. 344:783-792; Beniaminovitz et al. (2000) New Engl. J. Med. 342:613-619; Ghosh et al. (2003) New Engl. J. Med. 348:24-32; Lipsky et al. (2000) New Engl. J. Med. 343:1594-1602; Physicians' Desk Reference 2003 (Physicians' Desk Reference, 57th Ed); Medical Economics Company; ISBN: 1563634457; 57th edition (November 2002). Determination of the appropriate dose regimen may be made by the clinician, e.g., using parameters or factors known or suspected in the art to affect treatment or predicted to affect treatment, and will depend, for example, the individual's clinical history (e.g., previous therapy), the type and stage of the cancer to be treated and biomarkers of response to one or more of the therapeutic agents in the combination therapy.

Thus, the present disclosure contemplates embodiments of the combination therapy of the present disclosure that further includes a chemotherapy step comprising platinum-containing chemotherapy, pemetrexed and platinum chemotherapy or carboplatin and either paclitaxel or nab-paclitaxel. In particular embodiments, the combination therapy with a chemotherapy step may be used for treating at least NSCLC and HNSCC.

The combination therapy further in combination with a chemotherapy step may be used for the treatment any proliferative disease, in particular, treatment of cancer. In particular embodiments, the combination therapy of the present disclosure may be used to treat melanoma, non-small cell lung cancer, head and neck cancer, urothelial cancer, breast cancer, gastrointestinal cancer, multiple myeloma, hepatocellular cancer, non-Hodgkin lymphoma, renal cancer, Hodgkin lymphoma, mesothelioma, ovarian cancer, small cell lung cancer, esophageal cancer, anal cancer, biliary tract cancer, colorectal cancer, cervical cancer, thyroid cancer, or salivary cancer.

In another embodiment, the combination therapy further in combination with a chemotherapy step may be used to treat pancreatic cancer, bronchus cancer, prostate cancer, pancreatic cancer, stomach cancer, ovarian cancer, urinary bladder cancer, brain or central nervous system cancer, peripheral nervous system cancer, uterine or endometrial cancer, cancer of the oral cavity or pharynx, liver cancer, kidney cancer, testicular cancer, biliary tract cancer, small bowel or appendix cancer, adrenal gland cancer, osteosarcoma, chondrosarcoma, or cancer of hematological tissues.

In particular embodiments, the combination therapy with a chemotherapy step may be used to treat one or more cancers selected from melanoma (metastatic or unresectable), primary mediastinal large B-cell lymphoma (PMBCL), urothelial carcinoma, MSIHC, gastric cancer, cervical cancer, hepatocellular carcinoma (HCC), Merkel cell carcinoma (MCC), renal cell carcinoma (including advanced), and cutaneous squamous carcinoma.

### Combination Therapy Comprising A Human MSLN Binder And A Therapeutic Antibody

The human MSLN binder of the present disclosure may be administered in combination with one or more therapeutic agent, which is an antibody, for treatment of cancer or proliferative disease. The individual may undergo treatment with the therapeutic antibody at the same time the individual is undergoing the combination therapy of the present disclosure. The individual may undergo the combination therapy of the present disclosure after the individual has completed treatment with the therapeutic antibody. The individual may be administered the treatment with the therapeutic antibody after completion of the combination therapy. The combination therapy of the present disclosure may also be administered to an individual having recurrent or metastatic cancer with disease progression or relapse cancer and who is undergoing chemotherapy or who has completed chemotherapy. In particular embodiments, the therapeutic agent targets the programmed death 1 receptor or ligand., PD-1 and PD-L1, respectively.

Exemplary anti-PD-1 antibodies that may be used in a combination therapy with the human MSLN binder include any antibody that binds PD-1 and inhibits PD-1 from binding PD-L1. In a further embodiment, the exemplary anti-PD-1 antibody is selected from the group consisting of nivolumab, pembrolizumab, and cemiplimab-rwlc. Exemplary antibodies include the following anti-PD-1 antibodies and compositions comprising an anti-PD1 antibody and a pharmaceutically acceptable salt.

Pembrolizumab, also known as KEYTRUDA, lambrolizumab, MK-3475 or SCH-900475, is a humanized anti-PD-1 antibody described in U.S. Pat. No. 8,354,509 and WO2009/114335 and disclosed, e.g., in Hamid, et al., New England J. Med. 369 (2): 134-144 (2013).

Nivolumab, also known as OPDIVO, MDX-1106-04, ONO-4538, or BMS-936558, is a fully human IgG4 anti-PD-1 antibody described in WO2006/121168 and U.S. Pat. No. 8,008,449.

Cemiplimab-rwlc, also known as cemiplimab, LIBTAYO or REGN2810, is a recombinant human IgG4 monoclonal antibody that is described in WO2015112800 and U.S. Pat. No. 9,987,500.

In particular embodiments, the anti-PD-1 antibody comprises (i) a V_{H} comprising the three HC-CDRs of pembrolizumab fused or linked to an effector-silent HC constant domain and (ii) a V_{L} comprising the three LC-CDRs of pembrolizumab fused or linked to a LC kappa or lambda constant domain.

In particular embodiments, the anti-PD-1 antibody comprises (i) a V_{H} comprising the three HC-CDRs of nivolumab fused or linked to an effector-silent HC constant domain and (ii) a V_{L} comprising the three LC-CDRs of nivolumab fused or linked to a LC kappa or lambda constant domain.

In particular embodiments, the anti-PD-1 antibody comprises (i) a V_{H} comprising the three HC-CDRs of cemiplimab-rwlc fused or linked to an effector-silent HC constant domain and (ii) a V_{L} comprising the three LC-CDRs of nivolumab fused or linked to a LC kappa or lambda constant domain.

In particular embodiments, the anti-PD-1 antibody V_{H} may be fused or linked to an IgG1, IgG2, IgG3, or IgG4 HC constant domain that is not currently linked to the particular V_{H} or is linked to an IgG1, IgG2, IgG3, or IgG4 HC constant domain has been modified to include one or more mutations in the Fc domain that render the resulting anti-PD-1 antibody effecter-silent.

### Injection Device for Administering a Human MSLN Binder

The present disclosure also provides an injection device comprising a human MSLN binder as set forth herein or a pharmaceutical composition thereof. An injection device is a device that introduces a substance into the body of a patient via a parenteral route, e.g., intramuscular, subcutaneous or intravenous. For example, an injection device may be a syringe (e.g., pre-filled with the pharmaceutical composition, such as an auto-injector) which, for example, includes a cylinder or barrel for holding fluid to be injected (e.g., comprising the human MSLN binder or a pharmaceutical composition thereof), a needle for piecing skin and/or blood vessels for injection of the fluid; and a plunger for pushing the fluid out of the cylinder and through the needle bore. In an embodiment of the disclosure, an injection device that comprises a human MSLN binder or a pharmaceutical composition thereof is an intravenous (IV) injection device. Such a device includes the human MSLN binder or a pharmaceutical composition thereof in a cannula or trocar/needle which may be attached to a tube which may be attached to a bag or reservoir for holding fluid (e.g., saline; or lactated ringer solution comprising NaCl, sodium lactate, KCl, CaCl₂) and optionally including glucose) introduced into the body of the subject through the cannula or trocar/needle.

The human MSLN binder or a pharmaceutical composition thereof may, in an embodiment of the disclosure, be introduced into the device once the trocar and cannula are inserted into the vein of a subject and the trocar is removed from the inserted cannula. The IV device may, for example, be inserted into a peripheral vein (e.g., in the hand or arm); the superior vena cava or inferior vena cava, or within the right atrium of the heart (e.g., a central IV); or into a subclavian, internal jugular, or a femoral vein and, for example, advanced toward the heart until it reaches the superior vena cava or right atrium (e.g., a central venous line). In an embodiment of the disclosure, an injection device is an autoinjector; a jet injector or an external infusion pump. A jet injector uses a high-pressure narrow jet of liquid which penetrate the epidermis to introduce the human MSLN binder or a pharmaceutical composition thereof to a patient's body. External infusion pumps are medical devices that deliver the human MSLN binder or a pharmaceutical composition thereof into a patient's body in controlled amounts. External infusion pumps may be powered electrically or mechanically. Different pumps operate in different ways, for example, a syringe pump holds fluid in the reservoir of a syringe, and a moveable piston controls fluid delivery, an elastomeric pump holds fluid in a stretchable balloon reservoir, and pressure from the elastic walls of the balloon drives fluid delivery. In a peristaltic pump, a set of rollers pinches down on a length of flexible tubing, pushing fluid forward. In a multi-channel pump, fluids can be delivered from multiple reservoirs at multiple rates.

### Kits Comprising A Human MSLN Binder

Further provided are kits comprising one or more components that include, but are not limited to, a human MSLN binder, as discussed herein in association with one or more additional components including, but not limited to, a further therapeutic agent, as discussed herein. The human MSLN binder and/or the therapeutic agent can be formulated as a pure composition or in combination with a pharmaceutically acceptable carrier, in a pharmaceutical composition.

In one embodiment, the kit includes a human MSLN binder or a pharmaceutical composition thereof in one container (e.g., in a sterile glass or plastic vial) and a further therapeutic agent in another container (e.g., in a sterile glass or plastic vial).

In another embodiment, the kit comprises a combination of the disclosure, including a human MSLN binder or pharmaceutical composition thereof in combination with one or more therapeutic agents formulated together, optionally, in a pharmaceutical composition, in a single, common container.

If the kit includes a pharmaceutical composition for parenteral administration to a subject, the kit can include a device for performing such administration. For example, the kit can include one or more hypodermic needles or other injection devices as discussed above. Thus, the present disclosure includes a kit comprising an injection device and the human MSLN binder, e.g., wherein the injection device includes human MSLN binder or wherein the human MSLN binder is in a separate vessel.

The kit can include a package insert including information concerning the pharmaceutical compositions and dosage forms in the kit. Generally, such information aids patients and physicians in using the enclosed pharmaceutical compositions and dosage forms effectively and safely. For example, the following information regarding a combination of the disclosure may be supplied in the insert: pharmacokinetics, pharmacodynamics, clinical studies, efficacy parameters, indications and usage, contraindications, warnings, precautions, adverse reactions, overdosage, proper dosage and administration, how supplied, proper storage conditions, references, manufacturer/distributor information and patent information.

The following examples are intended to promote a further understanding of the present disclosure.

### General Methods

### Protein ELISAs

The wells of a 96-well plate are coated with 50 μL of purified human or rhesus MSLN in a carbonate coating buffer (5.3 g NaHCO₃ and 3.2 g Na₂CO₃ in 2 L H₂0) or HyClone Dulbecco's phosphate buffered saline (DPBS; Hyclone, cat #SH30028.02) at a concentration of 1-2 μg/mL and incubated overnight at 4° C. Afterwards, the plate wells are washed with well wash solution (phosphate buffered saline containing 0.05% polysorbate 20). The plate wells are then blocked by adding Super Block Blocking Buffer T20 (Thermo Scientific, #37536) for 1-2 hours at room temperature followed by washing with well wash solution. Next, 50 μL/well hybridoma supernatant fraction (used neat), or purified antibody starting at 10 μg/mL and serially diluted 4- or 5-fold in ELISA buffer (DPBS+0.1% BSA+0.05% polysorbate 20 is added to the wells. The plates are incubated for an hour at room temperature, then the wells washed with well wash solution and 50 μL/well horseradish peroxide (HRP) conjugated anti-species IgG diluted 1:3000-5000 in ELISA buffer is added to the wells. HRP conjugated anti-species IgG include goat anti-mouse IgG-HRP (Southern Biotech, cat #1030-05), goat anti-rat IgG-HRP (Southern Biotech, cat #3030-05), and goat anti-human IgG-HRP (Jackson Immunologics, cat #109-036-098). The plates are incubated for an hour at room temperature, then the wells washed with well wash and 50 μL/well ABTS Peroxidase Substrate (Kirkgaard & Perry Laboratories) is added to the wells. After 10 minutes the OD at 405 nm is measured using an ELISA plate reader.

### Cell ELISA on CHO Cells

The wells of a 96-well plate are seeded with sufficient cells to provide 95-100% confluency on day 0 (the day of assay). On day 0 the culture medium is removed and 50 μL/well hybridoma supernatant fraction, or purified antibody starting at 10 μg/mL or equal molar concentration and serially diluted 4 or 5-fold in CHO medium (DMEM F12, 10% Bovine serum). The plate is then incubated for an hour at room temperature or a temperature up to 37° C. Next, about 50 μL of a pre-determined concentration of ligand is added to the wells of the plate and the plate incubated for 30 minutes at room temperature or a temperature up to 37° C. The plate wells are washed with ELISA wash (PBS containing 0.05% polysorbate 20. Next, 50 μL/well of HRP-conjugated anti-species IgG diluted 1:2000 in CHO medium is added to the wells and the plate incubated for an hour at room temperature or a temperature up to 37° C. HRP conjugated anti-species IgG include goat anti-mouse IgG-HRP (Southern Biotech, cat #1030-05), goat anti-rat IgG-HRP (Southern Biotech, cat #3030-05), and goat anti-human IgG-HRP (Jackson Immunologics, cat #109-036-098). The plate wells are washed with the ELISA wash. Next, 50 μL/well TMB substrate (1 step Ultra TMB-ELISA: Thermo Fisher, Cat #34029) is added to the wells. When the blank starts to turn blue, 50 μL/well TMB stop solution (KPL, Cat #50-85-06) is added to the wells and A450-A620 is measured on an ELISA reader.

### Example 1

### Immunization and Screening for Human MSLN Binders

Trianni™ mice (AbCellera, Vancouver, BC) were immunized using mRNA encoding the full-length human mature MSLN. Splenocytes from the immunized animals were subsequently isolated and used for the fusion with the Sp2/0 myeloma partner to produce hybridomas. Hybridomas were then separately plated into 96-well plates to produce a hybridoma library and antibody supernatant fractions from the plated hybridomas were screened for binding to human MSLN, species cross-reactivity, affinity, binding to cells expressing full-length human MSLN, and binding to the human MSLN. Positive binding wells were selected for subcloning to ensure monoclonality. Monoclonal hybridomas that were obtained were then used for small scale antibody production and purification. Purified antibodies were subjected to another round of screening, including affinity, epitope binning, full length protein binding, human MSLN binding, protein, and cell binding, and competition assays.

The hybridoma clones SV017.13B12.1E1, SV017.47D7.1C2, SV017.50G3.1B1, SV017.51F4.1A1, SV018.20B12.1D1, and SV018.45B6.1G1 were selected for analysis.

### Example 2

### Antibody Production

ExpiCHO™ cells growing in suspension were transfected with antibody expression plasmids (HC+LC) using commercially available protocols and ExpiFectamine™ CHO reagents (ThermoFisher) (Sivasubramanian et al., MABS 9:29-42 (2017)). In brief, cells were transfected day 0 using 1 μg total DNA (3:2 ratio LC:HC) per 1 mL cells at a density of 6 million cells per mL and a viability >95% measured using a Vi-Cell (Beckman-Coulter). On day one, ExpiCHO™ feed and enhancer were added to the cell culture and the cell culture temperature was lowered to 32° C. On day five, a second EXPI-CHO™ feed was performed; cell viability was measured using a Vi-Cell (Beckman-Coulter). Cell cultures were harvested between day 8 and day 12 depending on a cell viability greater than 80% and the harvested cell culture fluid was centrifuged to remove cells and debris to provide a clarified supernatant fraction.

Antibodies were purified from clarified supernatant fraction using Protein A resin chromatography (mAbSelect Sure™ LX, GE Healthcare) as follows.

Protein A resin in a loading buffer was incubated with the clarified supernatant fraction overnight in 4° C. on a roller mixer. Afterwards, the protein A resin was collected from the clarified supernatant fraction and transferred to a chromatography column and washed with 10 column volumes (CV) of phosphate buffered saline (PBS). Antibody was then eluted from the chromatography column using an elution buffer comprising 20 mM sodium acetate, pH 3.5. One column volume (CV) fractions were collected and tested by Bradford assay to determine presence of protein. In some cases, Protein A purification was followed by anion exchange chromatography (Capto™ Q, GE Healthcare). Purified antibodies were buffer exchanged into a final formulation buffer comprising 20 mM sodium acetate, 9% sucrose, pH 5.5. Purified antibody was checked for purity by reduced and non-reduced capillary electrophoresis sodium dodecyl sulfate (CE-SDS) (PerkinElmer), concentration was measured by A280, and aggregate content was analyzed by SEC-UPLC (size exclusion ultra-performance liquid chromatography) using a BEH200 UPLC-SEC analytical column (Waters Corporation). Endotoxin was quantified using Endosafe® nexgen-MCS™ (Charles River). Intact mass was confirmed via Synapt® G2S QTOF or Xevo® G2 TOF (Waters).

The amino acid sequences for the antibodies produced by hybridoma clones SV017.13B12.1E1, SV017.47D7.1C2, SV017.50G3.1B1, SV017.51F4.1A1, SV018.20B12.1D1, and SV018.45B6.1G1 are shown in Table 6.

### Example 3

The antibodies produced by hybridoma clones SV017.13B12.1E1, SV017.47D7.1C2, SV017.50G3.1B1, SV017.51F4.1A1, SV018.20B12.1D1, and SV018.45B6.1G1 were evaluated by ELISA (enzyme-linked immunosorbent assay) for binding to human MSLN and non-human primate mesothelin (rhesus MSLN) both as soluble protein and membrane-bound protein expressed by Chinese hamster ovary K1 (CHOK1) cells genetically modified to express human MSLN or rhesus MSLN and the OVCAR3 cell line. The OVACAR3 cell line is a human ovarian cancer cell line that expresses high levels of human MSLN.

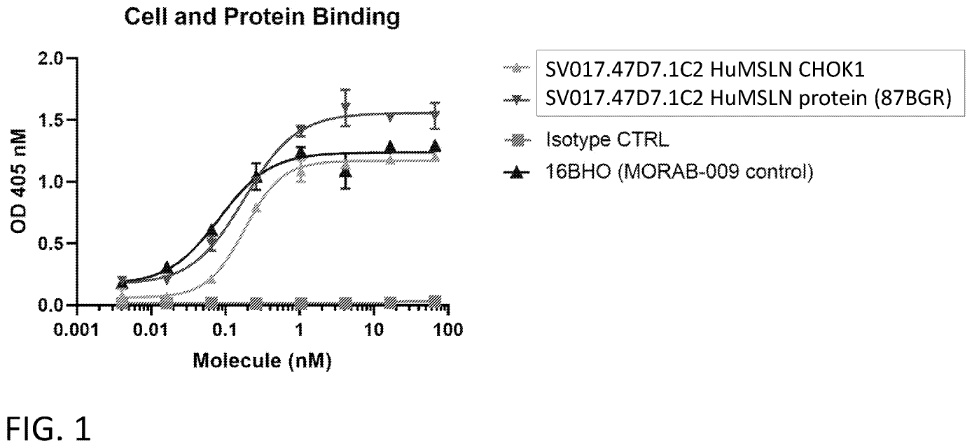

The results are presented in Table 5 and in FIGS. 1-3. As shown in Table 5, all the antibodies show strong binding to membrane-bound human MSLN (CHOK1 or OVCAR3). Antibodies identified from clones SV018.20B12.1D1 and SV018.45.1G1 show no detectable binding to rhesus MSLN and weak binding to soluble human MSLN and antibodies from clones SV017.13B12.1E1, SV017.47D7.1C2, SV017.50G3.1B1, and SV017.51F4.1A1 show weak binding to rhesus MSLN.

TABLE 5
Binding Characterization Data

| | | Rh | | | | Rh |
|---|---|---|---|---|---|---|
| | HuMSLN- | MSLN- | | OVCAR3 | HuMSLN | MSLN |
| | CHOK1 | CHOK1 | CHOK1 | Binding | (87BGR) | (87BIC) |
| | EC50 | EC50 | EC50 | EC50 | EC50 | EC50 |
| Antibody ID | μg/mL | μg/mL | μg/mL | μg/mL | μg/mL | μg/mL |
| SV017.13B12.1E1 | 0.02 | 0.23 | NB | 0.01 | 0.04 | 0.02 |
| SV017.47D7.1C2 | 0.03 | 0.26 | NB | 0.01 | 0.03 | 0.01 |
| SV017.50G3.1B1 | 0.05 | 0.33 | NB | 0.01 | 0.04 | 0.02 |
| SV017.51F4.1A1 | 0.04 | 0.26 | NB | 0.01 | 0.03 | 0.02 |
| SV018.20B12.1D1 | 0.01 | NB | NB | 0.01 | 0.11 | NB |
| SV018.45B6.1G1 | 0.01 | NB | NB | 0.00 | 1.37 | NB |

NB, means no binding
HuMSLN, means human MSLN
RhMSLN, means rhesus MSLN

FIGS. 1-3 show a comparison of the antibodies identified from hybridoma clones SV017.47D7.1C2, SV018.20B12.1D1, and SV018.45B6.1G1 compared to MORAB-009 as a positive control. MORAB-009 is an anti-human MSLN antibody having the nonproprietary name Amatuximab and comprising a heavy chain and light chain shown in SEQ ID NO: 40 and SEQ ID NO: 41, respectively. The data was generated from ELISAs.

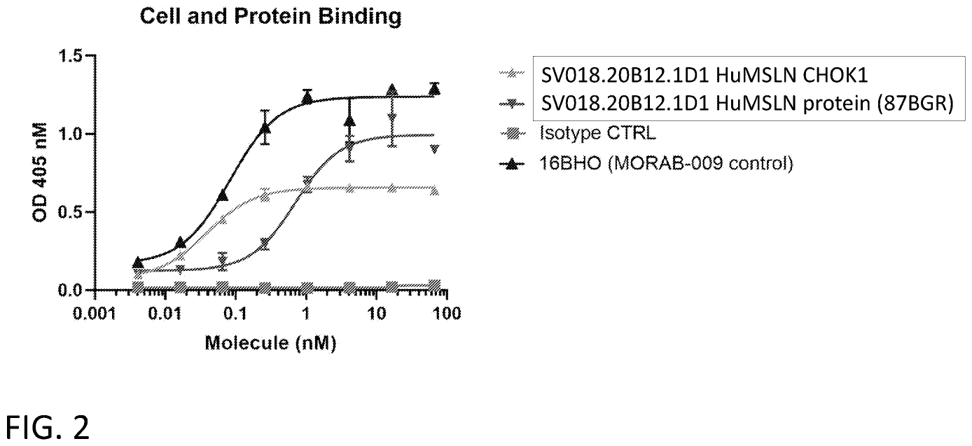

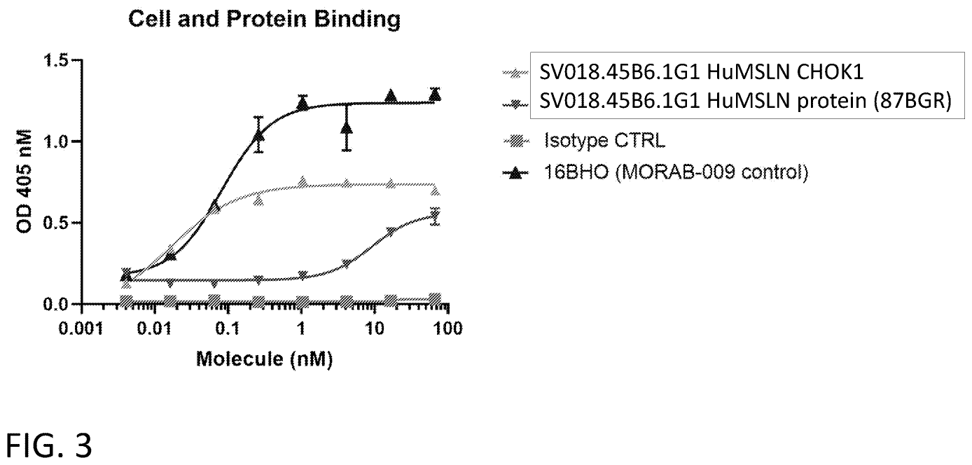

FIG. 1 shows that antibody SV017.47D7.1C2 could bind to membrane-bound human MSLN with a potency comparable to that of MORAB-009 and to soluble human MSLN with. a potency greater than the potency for binding membrane-bound human MSLN. FIG. 2 shows that antibody SV018.20B12.1D1 binds human MSLN with about half the potency displayed by MORAB-009 and bind soluble human MSLN with a potency less than it displays for binding membrane-bound human MSLN. FIG. 3 shows that antibody SV018.45B6.1G1 could bind human MSLN with about half the potency displayed by MORAB-009 and bind soluble human MSLN with a potency less than it displays for binding membrane-bound human MSLN.

The ELISA results show that the antibodies bind membrane-bound human MSLN strongly and membrane-bound rhesus MSLN weakly or not at all. The antibodies SV018.20B12.1D1 and SV018.45B6.1G1, which displayed no detectable binding to membrane bound rhesus MSLN, also displayed a lower potency for soluble human MSLN indicating that these antibodies have reduced ability to become a sink.

TABLE 6
Sequences

| SEQ ID | | |
|---|---|---|
| NO: | Description | Sequence |
| 1 | Human mesothelin | MALPTARPLLGSCGTPALGSLLFLLFSLGWVQPSRTLAG |
| | (precursor) | ETGQEAAPLDGVLANPPNISSLSPRQLLGFPCAEVSGLST |
| | Features: | ERVRELAVALAQKNVKLSTEQLRCLAHRLSEPPEDLDAL |
| | Amino acids 1-295 | PLDLLLFLNPDAFSGPQACTRFFSRITKANVDLLPRGAPE |
| | (MPF; megakaryocyte | RQRLLPAALACWGVRGSLLSEADVRALGGLACDLPGRF |
| | potentiating factor); | VAESAEVLLPRLVSCPGPLDQDQQEAARAALQGGGPPY |
| | Amino acids 296-622 | GPPSTWSVSTMDALRGLLPVLGQPIIRSIPQGIVAAWRQR |
| | mature protein | SSRDPSWRQPERTILRPRFRREVEKTACPSGKKAREIDES |
| | | LIFYKKWELEACVDAALLATQMDRVNAIPFTYEQLDVL |
| | | KHKLDELYPQGYPESVIQHLGYLFLKMSPEDIRKWNVTS |
| | | LETLKALLEVNKGHEMSPQVATLIDRFVKGRGQLDKDT |
| | | LDTLTAFYPGYLCSLSPEELSSVPPSSIWAVRPQDLDTCD |
| | | PRQLDVLYPKARLAFQNMNGSEYFVKIQSFLGGAPTEDL |
| | | KALSQQNVSMDLATFMKLRTDAVLPLTVAEVQKLLGPH |
| | | VEGLKAEERHRPVRDWILRQRQDDLDTLGLGLQGGIPN |
| | | GYLVLDLSMQEALSGTPCLLGPGPVLTVLALLLASTLA |
| 2 | SV017.47D7.1C2 VH | EVQLVESGGGLVQPGGSLRLSCAATGFTFSSYWMHWVR |
| | | QGPGKGLVWVSRINSDGTSTVYADSVKGRFTISRDNAK |
| | | NTLYLQMNSLRAEDTAVYYCARGRGIMTYWGQGTLVT |
| | | VSS |
| 3 | SV017.47D7.1C2 VL | DIQMTQSPSSLSASVGDRVTITCRASQDIGSWLTWYQRK |
| | | PEKAPKSLIYAASSLQSGVPSRFSGSGSGTDFTLTISSLQP |
| | | EDFATYYCQQYKSYPLTFGQGAKLEIK |
| 4 | SV017.47D7.1C2 VH- | SYWMH |
| | CDR1 Kabat | |
| 5 | SV017.47D7.1C2 VH- | RINSDGTSTVYADSVKG |
| | CDR2 Kabat | |
| 6 | SV017.47D7.1C2 VH- | GRGIMTY |
| | CDR3 Kabat | |
| 7 | SV017.47D7.1C2 VL- | RASQDIGSWLT |
| | CDR1 Kabat | |
| 8 | SV017.47D7.1C2 VL- | AASSLQS |
| | CDR2 Kabat | |
| 9 | SV017.47D7.1C2 VL- | QQYKSYPLT |
| | CDR3 Kabat | |
| 10 | SV018.20B12.1D1 VH | QVQLQESGPGLVKPSETLSLTCTVSGGSISSYYWSWIRQP |
| | | PGKGLEWIGYIYYSGSTNYNPSLKSRVTISVDTSKNQFSL |
| | | KLSSVTAADTAVYYCARTGYIPMDVWGQGTTVTVSS |
| 11 | SV018.20B12.1D1 VL | EIVLTQSPDFQSVTPKEKVTITCRASQSIGSNLHWYQQKP |
| | | DQSPKLLIKYVSQSFSGVPSRFSGSGSGTDFTLTINSLEAE |
| | | DAATYYCHQSRSLPYTFGQGTKLEIK |
| 12 | SV018.20B12.1D1 | SYYWS |
| | VH-CDR1 Kabat | |
| 13 | SV018.20B12.1D1 | YIYYSGSTNYNPSLKS |
| | VH-CDR2 Kabat | |
| 14 | SV018.20B12.1D1 | TGYIPMDV |
| | VH-CDR3 Kabat | |
| 15 | SV018.20B12.1D1 VL- | RASQSIGSNLH |
| | CDR1 Kabat | |
| 16 | SV018.20B12.1D1 VL- | YVSQSFS |
| | CDR2 Kabat | |
| 17 | SV018.20B12.1D1 VL- | HQSRSLPYT |
| | CDR3 Kabat | |
| 18 | SV018.45B6.1G1 VH | QVQLLQSGAEVKKPGASVKVSCKASGYTFTTYGISWVR |
| | | QAPGQGLEWLGWITAYNRNTNYAQKLQGRVTMTTDTS |
| | | TSTSFMVLRSLRSDDTAVYYCARVGDSDAFDIWGQGTM |
| | | VTVSS |
| 19 | SV018.45B6.1G1 VL | EIVMTQSPATLSLSPGERATLSCRTSQSVFNSFLSWYQQK |
| | | PGQAPRLLLYGTSTRATGIPARFSGSGSGTDFTLTISSLQP |
| | | EDFAVYYCQQDYKLPLTFGGGTKVEIK |
| 20 | SV018.45B6.1G1 VH- | TYGIS |
| | CDR1 Kabat | |
| 21 | SV018.45B6.1G1 VH- | WITAYNRNTNYAQKLQG |
| | CDR2 Kabat | |
| 22 | SV018.45B6.1G1 VH- | VGDSDAFDI |
| | CDR3 Kabat | |
| 23 | SV018.45B6.1G1 VL- | RTSQSVFNSFLS |
| | CDR1 Kabat | |
| 24 | SV018.45B6.1G1 VL- | GTSTRAT |
| | CDR2 Kabat | |
| 25 | SV018.45B6.1G1 VL- | QQDYKLPLT |
| | CDR3 Kabat | |
| 26 | SV017.13B12.1E1 VH | EVQLVESGGGLVQPGGSLRLSCAATGFTFSSYWMHWVR |
| | | QGPGKGLVWVSRINSDGTSTVYADSVKGRFTISRDNAK |
| | | NTLHLQMNSLRAEDTAVYYCARGRGIMTYWGQGTLVT |
| | | VSS |
| 27 | SV017.50G3.1B1 VH | EVQLVESGGGLVQPGESLRLSCAATGFTFSSYWMHWVR |
| | | QGPGKGLVWVSRINSDGTSTVYADSVKGRFTISRDNAK |
| | | NTLFLQMNSLRAEDTAVYYCARGRGIMTYWGQGTLVT |
| | | VSS |
| 28 | SV017.51F4.1A1 VH | EVQLVESGGGLVQPGGSLRLSCAATGFTFSSYWMHWVR |
| | | QGPGKGLVWVSRINSDGTSTVYADSVKGRFTISRDNAK |
| | | NTLYLQMNSLRAEDTAVYYCARGRGIMTYWGQGTLVT |
| | | VSS |
| 29 | Human IgG₁ HC | ASTKGPSVFPLAPSSKSTSGGTAALGCLVKDYFPEPVTVS |
| | constant domain | WNSGALTSGVHTFPAVLQSSGLYSLSSVVTVPSSSLGTQ |
| | | TYICNVNHKPSNTKVDKKVEPKSCDKTHTCPPCPAPELL |
| | | GGPSVFLFPPKPKDTLMISRTPEVTCVVVDVSHEDPEVKF |
| | | NWYVDGVEVHNAKTKPREEQYNSTYRVVSVLTVLHQD |
| | | WLNGKEYKCKVSNKALPAPIEKTISKAKGQPREPQVYTL |
| | | PPSRDELTKNQVSLTCLVKGFYPSDIAVEWESNGQPENN |
| | | YKTTPPVLDSDGSFFLYSKLTVDKSRWQQGNVFSCSVM |
| | | HEALHNHYTQKSLSLSPGK |
| 30 | Human IgG₁ HC | ASTKGPSVFPLAPSSKSTSGGTAALGCLVKDYFPEPVTVS |
| | Constant domain | WNSGALTSGVHTFPAVLQSSGLYSLSSVVTVPSSSLGTQ |
| | (E233A/L235A) | TYICNVNHKPSNTKVDKKVEPKSCDKTHTCPPCPAPALA |
| | | GGPSVFLFPPKPKDTLMISRTPEVTCVVVDVSHEDPEVKF |
| | | NWYVDGVEVHNAKTKPREEQYNSTYRVVSVLTVLHQD |
| | | WLNGKEYKCKVSNKALPAPIEKTISKAKGQPREPQVYTL |
| | | PPSRDELTKNQVSLTCLVKGFYPSDIAVEWESNGQPENN |
| | | YKTTPPVLDSDGSFFLYSKLTVDKSRWQQGNVFSCSVM |
| | | HEALHNHYTQKSLSLSPGK |
| 31 | Human IgG₁ HC | ASTKGPSVFPLAPSSKSTSGGTAALGCLVKDYFPEPVTVS |
| | Constant domain | WNSGALTSGVHTFPAVLQSSGLYSLSSVVTVPSSSLGTQ |
| | (L234A L235A | TYICNVNHKPSNTKVDKKVEPKSCDKTHTCPPCPAPEAA |
| | D265S) | GGPSVFLFPPKPKDTLMISRTPEVTCVVVSVSHEDPEVKF |
| | | NWYVDGVEVHNAKTKPREEQYNSTYRVVSVLTVLHQD |
| | | WLNGKEYKCKVSNKALPAPIEKTISKAKGQPREPQVYTL |
| | | PPSRDELTKNQVSLTCLVKGFYPSDIAVEWESNGQPENN |
| | | YKTTPPVLDSDGSFFLYSKLTVDKSRWQQGNVFSCSVM |
| | | HEALHNHYTQKSLSLSPGK |
| 32 | Human IgG₁ HC | ASTKGPSVFPLAPSSKSTSGGTAALGCLVKDYFPEPVTVS |
| | Constant domain | WNSGALTSGVHTFPAVLQSSGLYSLSSVVTVPSSSLGTQ |
| | (L234A L235A | TYICNVNHKPSNTKVDKKVEPKSCDKTHTCPPCPAPEAA |
| | P329G) | GGPSVFLFPPKPKDTLMISRTPEVTCVVVSVSHEDPEVKF |
| | | NWYVDGVEVHNAKTKPREEQYNSTYRVVSVLTVLHQD |
| | | WLNGKEYKCKVSNKALGAPIEKTISKAKGQPREPQVYT |
| | | LPPSRDELTKNQVSLTCLVKGFYPSDIAVEWESNGQPEN |
| | | NYKTTPPVLDSDGSFFLYSKLTVDKSRWQQGNVFSCSV |
| | | MHEALHNHYTQKSLSLSPGK |
| 33 | Human IgG₁ HC | ASTKGPSVFPLAPSSKSTSGGTAALGCLVKDYFPEPVTVS |
| | Constant domain | WNSGALTSGVHTFPAVLQSSGLYSLSSVVTVPSSSLGTQ |
| | (L235E) | TYICNVNHKPSNTKVDKKVEPKSCDKTHTCPPCPAPELE |
| | | GGPSVFLFPPKPKDTLMISRTPEVTCVVVDVSHEDPEVKF |
| | | NWYVDGVEVHNAKTKPREEQYNSTYRVVSVLTVLHQD |
| | | WLNGKEYKCKVSNKALPAPIEKTISKAKGQPREPQVYTL |
| | | PPSRDELTKNQVSLTCLVKGFYPSDIAVEWESNGQPENN |
| | | YKTTPPVLDSDGSFFLYSKLTVDKSRWQQGNVFSCSVM |
| | | HEALHNHYTQKSLSLSPGK |
| 34 | Human IgG₁ HC | ASTKGPSVFPLAPSSKSTSGGTAALGCLVKDYFPEPVTVS |
| | Constant domain | WNSGALTSGVHTFPAVLQSSGLYSLSSVVTVPSSSLGTQ |
| | (D265A) | TYICNVNHKPSNTKVDKKVEPKSCDKTHTCPPCPAPELL |
| | | GGPSVFLFPPKPKDTLMISRTPEVTCVVVAVSHEDPEVKF |
| | | NWYVDGVEVHNAKTKPREEQYNSTYRVVSVLTVLHQD |
| | | WLNGKEYKCKVSNKALPAPIEKTISKAKGQPREPQVYTL |
| | | PPSRDELTKNQVSLTCLVKGFYPSDIAVEWESNGQPENN |
| | | YKTTPPVLDSDGSFFLYSKLTVDKSRWQQGNVFSCSVM |
| | | HEALHNHYTQKSLSLSPGK |
| 35 | Human IgG₁ HC | ASTKGPSVFPLAPSSKSTSGGTAALGCLVKDYFPEPVTVS |
| | Constant domain | WNSGALTSGVHTFPAVLQSSGLYSLSSVVTVPSSSLGTQ |
| | (D265A N297G) | TYICNVNHKPSNTKVDKKVEPKSCDKTHTCPPCPAPELL |
| | | GGPSVFLFPPKPKDTLMISRTPEVTCVVVAVSHEDPEVKF |
| | | NWYVDGVEVHNAKTKPREEQYGSTYRVVSVLTVLHQD |
| | | WLNGKEYKCKVSNKALPAPIEKTISKAKGQPREPQVYTL |
| | | PPSRDELTKNQVSLTCLVKGFYPSDIAVEWESNGQPENN |
| | | YKTTPPVLDSDGSFFLYSKLTVDKSRWQQGNVFSCSVM |
| | | HEALHNHYTQKSLSLSPGK |
| 36 | Human IgG₁ HC | ASTKGPSVFPLAPSSKSTSGGTAALGCLVKDYFPEPVTVS |
| | Constant domain | WNSGALTSGVHTFPAVLQSSGLYSLSSVVTVPSSSLGTQ |
| | (N297X, wherein X is | TYICNVNHKPSNTKVDKKVEPKSCDKTHTCPPCPAPELL |
| | any amino acid other | GGPSVFLFPPKPKDTLMISRTPEVTCVVVDVSHEDPEVKF |
| | than N) | NWYVDGVEVHNAKTKPREEQYXSTYRVVSVLTVLHQD |
| | | WLNGKEYKCKVSNKALPAPIEKTISKAKGQPREPQVYTL |
| | | PPSRDELTKNQVSLTCLVKGFYPSDIAVEWESNGQPENN |
| | | YKTTPPVLDSDGSFFLYSKLTVDKSRWQQGNVFSCSVM |
| | | HEALHNHYTQKSLSLSPGK |
| 37 | Human IgG₁ HC | ASTKGPSVFPLAPSSKSTSGGTAALGCLVKDYFPEPVTVS |
| | Constant domain | WNSGALTSGVHTFPAVLQSSGLYSLSSVVTVPSSSLGTQ |
| | (N297A/D356E/L358M) | TYICNVNHKPSNTKVDKKVEPKSCDKTHTCPPCPAPELL |
| | | GGPSVFLFPPKPKDTLMISRTPEVTCVVVDVSHEDPEVKF |
| | | NWYVDGVEVHNAKTKPREEQYASTYRVVSVLTVLHQD |
| | | WLNGKEYKCKVSNKALPAPIEKTISKAKGQPREPQVYTL |
| | | PPSREEMTKNQVSLTCLVKGFYPSDIAVEWESNGQPENN |
| | | YKTTPPVLDSDGSFFLYSKLTVDKSRWQQGNVFSCSVM |
| | | HEALHNHYTQKSLSLSPGK |
| 38 | Human LC Kappa | RTVAAPSVFIFPPSDEQLKSGTASVVCLLNNFYPREAKV |
| | constant domain | QWKVDNALQSGNSQESVTEQDSKDSTYSLSSTLTLSKA |
| | | DYEKHKVYACEVTHQGLSSPVTKSFNRGEC |
| 39 | Rhesus mesothelin | MALPMARPLSGSCGTPAVGSLLFLLFSLGWVQPSRVLA |
| | precursor | GETRQEAAPLDGILTNAPDIASLSPRQLLGFTCVEVSGLS |
| | Features: | TELVQELAVALGQKNVKLSAEQLRCLAHRLSEPPEDLAL |
| | Amino acids 1-294 | PLDLLLFLNPDAFSGPQACTHFFSRVAKANVDLLPRGAP |
| | (MPF; megakaryocyte | ERQRLLPAALTCWGVRGSLLSEADVRALGGLACDLPGC |
| | potentiating factor); | FVAESAEVVLPRLVRCLGPLDQDQQEAARAALQRGGPP |
| | Amino acids 295-621 | YGPPSTWSISTLDDLQSLLPVLGQPVIHSIPKGILAAWRQ |
| | mature protein | RSSRDPSWQQPEQTVLRPRFRRDVERTTCPPEKEVHEIDE |
| | | SLIFYKKRELEACVDAALLAAQMDRVDAIPFTYEQLDVL |
| | | KHKLDELYPQGYPESVIRHLGHLFLKMSPEDIRKWNVTS |
| | | LETLKALLKVSKGHEMSAQVATLIDRVVVGRGQLDKDT |
| | | VDTLTAFCPGCLCSLSPERLSSVPPSVIGAVRPQDLDTCG |
| | | PRQLDVLYPKARLAFQNMSGSEYFVKIRPFLGGAPTEDV |
| | | KALSQQNVSMDLATFMKLRREAVLPLTVAEVQKLLGPH |
| | | VEGLKVEEQHSPVRDWILKQRQDDLDTLGLGLQGGIPN |
| | | GYLILDLSVREALSGTPCLLGPGPVLTVLALLLASTLA |
| 40 | MORAB-009 Heavy | QVQLQQSGPELEKPGASVKISCKASGYSFTGYTMNWVK |
| | chain | QSHGKSLEWIGLITPYNGASSYNQKFRGKATLTVDKSSS |
| | | TAYMDLLSLTSEDSAVYFCARGGYDGRGFDYWGSGTPV |
| | | TVSSASTKGPSVFPLAPSSKSTSGGTAALGCLVKDYFPEP |
| | | VTVSWNSGALTSGVHTFPAVLQSSGLYSLSSVVTVPSSS |
| | | LGTQTYICNVNHKPSNTKVDKKVEPKSCDKTHTCPPCPA |
| | | PELLGGPSVFLFPPKPKDTLMISRTPEVTCVVVDVSHEDP |
| | | EVKFNWYVDGVEVHNAKTKPREEQYNSTYRVVSVLTV |
| | | LHQDWLNGKEYKCKVSNKALPAPIEKTISKAKGQPREPQ |
| | | VYTLPPSRDELTKNQVSLTCLVKGFYPSDIAVEWESNGQ |
| | | PENNYKTTPPVLDSDGSFFLYSKLTVDKSRWQQGNVFSC |
| | | SVMHEALHNHYTQKSLSLSPGK |
| 41 | MORAB-009 light | DIELTQSPAIMSASPGEKVTMTCSASSSVSYMHWYQQKS |
| | chain | GTSPKRWIYDTSKLASGVPGRFSGSGSGNSYSLTISSVEA |
| | | EDDATYYCQQWSKHPLTFGSGTKVEIKRTVAAPSVFIFP |
| | | PSDEQLKSGTASVVCLLNNFYPREAKVQWKVDNALQSG |
| | | NSQESVTEQDSKDSTYSLSSTLTLSKADYEKHKVYACEV |
| | | THQGLSSPVTKSFNRGEC |

While the present disclosure is described herein with reference to illustrated embodiments, it should be understood that the disclosure is not limited hereto. Those having ordinary skill in the art and access to the teachings herein will recognize additional modifications and embodiments within the scope thereof. Therefore, the present disclosure is limited only by the claims attached herein.

What is claimed:

1. A human mesothelin (MSLN) binder comprising:
(a) the six complementarity determining regions (CDRs) of an antibody comprising a heavy chain variable domain (V_{H}) comprising the amino acid sequence set forth in SEQ ID NO: 18 and a light chain variable domain (V_{L}) comprising the amino acid sequence set forth in SEQ ID NO: 19;
(b) the six CDRs of an antibody comprising a V_{H} comprising the amino acid sequence set forth in SEQ ID NO: 10 and a V_{L} comprising the amino acid sequence set forth in SEQ ID NO: 11; or,
(c) the six CDRs of an antibody comprising a V_{H} comprising the amino acid sequence set forth in SEQ ID NO: 2, 26, 27, or 28 and a V_{L} comprising the amino acid sequence set forth in SEQ ID NO: 3,
wherein in (a), (b), or (c), the CDRs are defined using the Kabat, Chothia, AbM, ImMunoGeneTics (IMGT), or Contact numbering scheme.

2. The human MSLN binder of claim 1, wherein the V_{H} comprises a CDR 1 comprising the amino acid sequence set forth in SEQ ID NO: 4, a CDR 2 comprising the amino acid sequence set forth in SEQ ID NO: 5, and a CDR 3 comprising the amino acid sequence set forth in SEQ ID NO:6; and, the V_{L} comprises a CDR 1 comprising the amino acid sequence set forth in SEQ ID NO: 7, a CDR 2 comprising the amino acid sequence set forth in SEQ ID NO: 8, and a CDR 3 comprising the amino acid sequence set forth in SEQ ID NO: 9.

3. The human MSLN binder of claim 1, wherein the V_{H} comprises a CDR 1 comprising the amino acid sequence set forth in SEQ ID NO: 12, a CDR 2 comprising the amino acid sequence set forth in SEQ ID NO: 13, and a CDR 3 comprising the amino acid sequence set forth in SEQ ID NO:14; and, the V_{L} comprises a CDR 1 comprising the amino acid sequence set forth in SEQ ID NO: 15, a CDR 2 comprising the amino acid sequence set forth in SEQ ID NO: 16, and a CDR 3 comprising the amino acid sequence set forth in SEQ ID NO: 17.

4. The human MSLN binder of claim 1, wherein the V_{H} comprises a CDR 1 comprising the amino acid sequence set forth in SEQ ID NO: 20, a CDR 2 comprising the amino acid sequence set forth in SEQ ID NO: 21, and a CDR 3 comprising the amino acid sequence set forth in SEQ ID NO: 22; and, the V_{L} comprises a CDR 1 comprising the amino acid sequence set forth in SEQ ID NO: 23, a CDR 2 comprising the amino acid sequence set forth in SEQ ID NO: 24, and a CDR 3 comprising the amino acid sequence set forth in SEQ ID NO: 25.

5. The human MSLN binder of claim 1, wherein the human MSLN binder comprises a V_{H} comprising
(i) the amino acid sequence set forth in SEQ ID NO: 2 or an amino acid sequence having the 2nd amino acid residue to the 116th amino acid residue of the amino acid sequence ranging from the 2nd amino acid residue to the 116th amino acid residue set forth in SEQ ID NO: 2 and the 1st amino acid residue of the amino acid sequence being pyroglutamate;
(ii) the amino acid sequence set forth in SEQ ID NO: 26 or an amino acid sequence having the 2nd amino acid residue to the 116th amino acid residue of the amino acid sequence ranging from the 2nd amino acid residue to the 116th amino acid residue set forth in SEQ ID NO: 26 and the 1st amino acid residue of the amino acid sequence being pyroglutamate;
(iii) the amino acid sequence set forth in SEQ ID NO: 27 or an amino acid sequence having the 2nd amino acid residue to the 116th amino acid residue of the amino acid sequence ranging from the 2nd amino acid residue to the 116th amino acid residue set forth in SEQ ID NO: 27 and the 1st amino acid residue of the amino acid sequence being pyroglutamate; or
(iii) the amino acid sequence set forth in SEQ ID NO: 28 or an amino acid sequence having the 2nd amino acid residue to the 116th amino acid residue of the amino acid sequence ranging from the 2nd amino acid residue to the 116th amino acid residue set forth in SEQ ID NO: 28 and the 1st amino acid residue of the amino acid sequence being pyroglutamate, and
a V_{L} comprising the amino acid sequence set forth in SEQ ID NO: 3.

6. The human MSLN binder of claim 5, comprising a V_{H} comprising the amino acid sequence set forth in SEQ ID NO: 2; and a V_{L} comprising the amino acid sequence set forth in SEQ ID NO: 3.

7. The human MSLN binder of claim 5, comprising a V_{H} comprising an amino acid sequence having the 2nd amino acid residue to the 116th amino acid residue of the amino acid sequence ranging from the 2nd amino acid residue to the 116th amino acid residue set forth in SEQ ID NO: 2 and the 1st amino acid residue of the amino acid sequence being pyroglutamate; and a V_{L} comprising the amino acid sequence set forth in SEQ ID NO: 3.

8. The human MSLN binder of claim 5, comprising a V_{H} comprising the amino acid sequence set forth in SEQ ID NO: 26; and a V_{L} comprising the amino acid sequence set forth in SEQ ID NO: 3.

9. The human MSLN binder of claim 5, comprising a V_{H} comprising an amino acid sequence having the 2nd amino acid residue to the 116th amino acid residue of the amino acid sequence ranging from the 2nd amino acid residue to the 116th amino acid residue set forth in SEQ ID NO: 26 and the 1st amino acid residue of the amino acid sequence being pyroglutamate; and a V_{L} comprising the amino acid sequence set forth in SEQ ID NO: 3.

10. The human MSLN binder of claim 5, comprising a V_{H} comprising the amino acid sequence set forth in SEQ ID NO: 27; and a V_{L} comprising the amino acid sequence set forth in SEQ ID NO: 3.

11. The human MSLN binder of claim 5, comprising a V_{H} comprising an amino acid sequence having the 2nd amino acid residue to the 116th amino acid residue of the amino acid sequence ranging from the 2nd amino acid residue to the 116th amino acid residue set forth in SEQ ID NO: 27 and the 1st amino acid residue of the amino acid sequence being pyroglutamate; and a V_{L} comprising the amino acid sequence set forth in SEQ ID NO: 3.

12. The human MSLN binder of claim 5, comprising a V_{H} comprising the amino acid sequence set forth in SEQ ID NO: 28; and a V_{L} comprising the amino acid sequence set forth in SEQ ID NO: 3.

13. The human MSLN binder of claim 5, comprising a V_{H} comprising an amino acid sequence having the 2nd amino acid residue to the 116th amino acid residue of the amino acid sequence ranging from the 2nd amino acid residue to the 116th amino acid residue set forth in SEQ ID NO: 28 and the 1st amino acid residue of the amino acid sequence being pyroglutamate; and a V_{L} comprising the amino acid sequence set forth in SEQ ID NO: 3.

14. The human MSLN binder of claim 1, wherein the human MSLN binder comprises a V_{H} comprising the amino acid sequence set forth in SEQ ID NO: 10 or an amino acid sequence having the 2nd amino acid residue to the 116th amino acid residue of the amino acid sequence ranging from the 2nd amino acid residue to the 116th amino acid residue set forth in SEQ ID NO: 10 and the 1st amino acid residue of the amino acid sequence being pyroglutamate, and
a V_{L} comprising the amino acid sequence set forth in SEQ ID NO: 11 or an amino acid sequence having the 2nd amino acid residue to the 107th amino acid residue of the amino acid sequence ranging from the 2nd amino acid residue to the 107th amino acid residue set forth in SEQ ID NO: 11 and the 1st amino acid residue of the amino acid sequence being pyroglutamate.

15. The human MSLN binder of claim 14, comprising a V_{H} comprising the amino acid sequence set forth in SEQ ID NO: 10; and a V_{L} comprising the amino acid sequence set forth in SEQ ID NO: 11.

16. The human MSLN binder of claim 14, comprising a V_{H} comprising an amino acid sequence having the 2nd amino acid residue to the 116th amino acid residue of the amino acid sequence ranging from the 2nd amino acid residue to the 116th amino acid residue set forth in SEQ ID NO: 10 and the 1st amino acid residue of the amino acid sequence being pyroglutamate; and a V_{L} comprising the amino acid sequence set forth in SEQ ID NO: 11.

17. The human MSLN binder of claim 14, comprising a V_{H} comprising the amino acid sequence set forth in SEQ ID NO: 10; and a V_{L} comprising an amino acid sequence having the 2nd amino acid residue to the 107th amino acid residue of the amino acid sequence ranging from the 2nd amino acid residue to the 107th amino acid residue set forth in SEQ ID NO: 11 and the 1st amino acid residue of the amino acid sequence being pyroglutamate.

18. The human MSLN binder of claim 14, comprising a V_{H} comprising an amino acid sequence having the 2nd amino acid residue to the 116th amino acid residue of the amino acid sequence ranging from the 2nd amino acid residue to the 116th amino acid residue set forth in SEQ ID NO: 10 and the 1st amino acid residue of the amino acid sequence being pyroglutamate; and a V_{L} comprising an amino acid sequence having the 2nd amino acid residue to the 107th amino acid residue of the amino acid sequence ranging from the 2nd amino acid residue to the 107th amino acid residue set forth in SEQ ID NO: 11 and the 1st amino acid residue of the amino acid sequence being pyroglutamate.

19. The human MSLN binder of claim 1, wherein the human MSLN binder comprises a V_{H} comprising the amino acid sequence set forth in SEQ ID NO: 18 or an amino acid sequence having the 2nd amino acid residue to the 118th amino acid residue of the amino acid sequence ranging from the 2nd amino acid residue to the 118th amino acid residue set forth in SEQ ID NO: 18 and the 1st amino acid residue of the amino acid sequence being pyroglutamate, and
a V_{L} comprising the amino acid sequence set forth in SEQ ID NO: 19 or an amino acid sequence having the 2nd amino acid residue to the 108th amino acid residue of the amino acid sequence ranging from the 2nd amino acid residue to the 108th amino acid residue set forth in SEQ ID NO: 19 and the 1st amino acid residue of the amino acid sequence being pyroglutamate.

20. The human MSLN binder of claim 19, comprising a V_{H} comprising the amino acid sequence set forth in SEQ ID NO: 18; and a V_{L} comprising the amino acid sequence set forth in SEQ ID NO: 19.

21. The human MSLN binder of claim 19, comprising a V_{H} comprising an amino acid sequence having the 2nd amino acid residue to the 118th amino acid residue of the amino acid sequence ranging from the 2nd amino acid residue to the 118th amino acid residue set forth in SEQ ID NO: 18 and the 1st amino acid residue of the amino acid sequence being pyroglutamate; and a V_{L} comprising the amino acid sequence set forth in SEQ ID NO: 19.

22. The human MSLN binder of claim 19, comprising a V_{H} comprising the amino acid sequence set forth in SEQ ID NO: 18; and a V_{L} comprising an amino acid sequence having the 2nd amino acid residue to the 108th amino acid residue of the amino acid sequence ranging from the 2nd amino acid residue to the 108th amino acid residue set forth in SEQ ID NO: 19 and the 1st amino acid residue of the amino acid sequence being pyroglutamate.

23. The human MSLN binder of claim 19, comprising a V_{H} comprising an amino acid sequence having the 2nd amino acid residue to the 118th amino acid residue of the amino acid sequence ranging from the 2nd amino acid residue to the 118th amino acid residue set forth in SEQ ID NO: 18 and the 1st amino acid residue of the amino acid sequence being pyroglutamate; and a V_{L} comprising an amino acid sequence having the 2nd amino acid residue to the 108th amino acid residue of the amino acid sequence ranging from the 2nd amino acid residue to the 108th amino acid residue set forth in SEQ ID NO: 19 and the 1st amino acid residue of the amino acid sequence being pyroglutamate.

24. The human MSLN binder of claim 1, wherein the human MSLN binder comprises a V_{H} comprising an amino acid sequence with at least 90% identity to the amino acid sequence set forth in SEQ ID NO: 2, SEQ ID NO: 26, SEQ ID NO: 27, or SEQ ID NO: 28; and a V_{L} comprising an amino acid sequence with at least 90% identity to the amino acid sequence set forth in SEQ ID NO: 3, with the proviso that the V_{H} comprises a CDR 1 comprising the amino acid sequence set forth in SEQ ID NO: 4, a CDR 2 comprising the amino acid sequence set forth in SEQ ID NO: 5, and a CDR 3 comprising the amino acid sequence set forth in SEQ ID NO:6; and, the V_{L} comprises a CDR 1 comprising the amino acid sequence set forth in SEQ ID NO: 7, a CDR 2 comprising the amino acid sequence set forth in SEQ ID NO: 8, and a CDR 3 comprising the amino acid sequence set forth in SEQ ID NO: 9.

25. The human MSLN binder of claim 1, wherein the human MSLN binder comprises a V_{H} comprising an amino acid sequence with at least 90% identity to the amino acid sequence set forth in SEQ ID NO: 10; and a V_{L} comprising an amino acid sequence with at least 90% identity to the amino acid sequence set forth in SEQ ID NO: 11, with the proviso that the V_{H} comprises a CDR 1 comprising the amino acid sequence set forth in SEQ ID NO: 12, a CDR 2 comprising the amino acid sequence set forth in SEQ ID NO: 13, and a CDR 3 comprising the amino acid sequence set forth in SEQ ID NO: 14; and, the V_{L} comprises a CDR 1 comprising the amino acid sequence set forth in SEQ ID NO: 15, a CDR 2 comprising the amino acid sequence set forth in SEQ ID NO: 16, and a CDR 3 comprising the amino acid sequence set forth in SEQ ID NO: 17.

26. The human MSLN binder of claim 1, wherein the human MSLN binder comprises a V_{H} comprising an amino acid sequence with at least 90% identity to the amino acid sequence set forth in SEQ ID NO: 18; and a V_{L} comprising an amino acid sequence with at least 90% identity to the amino acid sequence set forth in SEQ ID NO: 19, with the proviso the V_{H} comprises a CDR 1 comprising the amino acid sequence set forth in SEQ ID NO: 20, a CDR 2 comprising the amino acid sequence set forth in SEQ ID NO: 21, and a CDR 3 comprising the amino acid sequence set forth in SEQ ID NO:22; and, the V_{L} comprises a CDR 1 comprising the amino acid sequence set forth in SEQ ID NO: 23, a CDR 2 comprising the amino acid sequence set forth in SEQ ID NO: 24, and a CDR 3 comprising the amino acid sequence set forth in SEQ ID NO: 25.

27. The human MSLN binder of claim 1, wherein the human MSLN binder comprises an antibody comprising a heavy chain constant domain of the IgG1 isotype and a light chain constant domain of the human kappa or human lambda isotype.

28. The human MSLN binder of claim 27, wherein the heavy chain constant domain comprises the amino acid sequence set forth in SEQ ID NO: 29 or an amino acid sequence having the 1st amino acid residue to the 329th amino acid residue of the amino acid sequence ranging from the 1st amino acid residue to the 329th amino acid residue set forth in SEQ ID NO: 29.

29. The human MSLN binder of claim 27, wherein the heavy chain constant domain of the IgG1 isotype comprises an Fc domain comprising one or more mutations that render the constant domain effector-silent.

30. The human MSLN binder of claim 29, wherein the effector-silent constant domain comprises
(i) the amino acid sequence set forth in SEQ ID NO: 30 or an amino acid sequence having the 1st amino acid residue to the 329th amino acid residue of the amino acid sequence ranging from the 1st amino acid residue to the 329th amino acid residue set forth in SEQ ID NO: 30;
(ii) the amino acid sequence set forth in SEQ ID NO: 31 or an amino acid sequence having the 1st amino acid residue to the 329th amino acid residue of the amino acid sequence ranging from the 1st amino acid residue to the 329th amino acid residue set forth in SEQ ID NO: 31;
(iii) the amino acid sequence set forth in SEQ ID NO: 32 or an amino acid sequence having the 1st amino acid residue to the 329th amino acid residue of the amino acid sequence ranging from the 1st amino acid residue to the 329th amino acid residue set forth in SEQ ID NO: 32;
(iv) the amino acid sequence set forth in SEQ ID NO: 33 or an amino acid sequence having the 1st amino acid residue to the 329th amino acid residue of the amino acid sequence ranging from the 1st amino acid residue to the 329th amino acid residue set forth in SEQ ID NO: 33;
(v) the amino acid sequence set forth in SEQ ID NO: 34 or an amino acid sequence having the 1st amino acid residue to the 329th amino acid residue of the amino acid sequence ranging from the 1st amino acid residue to the 329th amino acid residue set forth in SEQ ID NO: 34;
(vi) the amino acid sequence set forth in SEQ ID NO: 35 or an amino acid sequence having the 1st amino acid residue to the 329th amino acid residue of the amino acid sequence ranging from the 1st amino acid residue to the 329th amino acid residue set forth in SEQ ID NO: 35;
(vii) the amino acid sequence set forth in SEQ ID NO: 36 or an amino acid sequence having the 1st amino acid residue to the 329th amino acid residue of the amino acid sequence ranging from the 1st amino acid residue to the 329th amino acid residue set forth in SEQ ID NO: 36; or
(viii) the amino acid sequence set forth in SEQ ID NO: 37 or an amino acid sequence having the 1st amino acid residue to the 329th amino acid residue of the amino acid sequence ranging from the 1st amino acid residue to the 329th amino acid residue set forth in SEQ ID NO: 37.

31. The human MSLN binder of claim 30, wherein the effector-silent constant domain comprises the amino acid sequence set forth in SEQ ID NO: 31 or an amino acid sequence having the 1st amino acid residue to the 329th amino acid residue of the amino acid sequence ranging from the 1st amino acid residue to the 329th amino acid residue set forth in SEQ ID NO: 31.

32. The human MSLN binder of claim 27, wherein the light chain constant domain comprises the human kappa isotype comprising the amino acid sequence set forth in SEQ ID NO: 38.

33. The human MSLN binder of claim 1, wherein the human MSLN binder comprises a Fab fragment, a Fab′ fragment, a F(ab′)₂ fragment, an Fv region, or an ScFv.

34. A composition comprising the human MSLN binder of any one of claims 1-33 and a pharmaceutically acceptable carrier or diluent.

35. A method for treating cancer or proliferative disease in an individual in need of the treatment comprising administering to the individual a therapeutically effective amount of a human MSLN binder of any one of claims 1-33 or the composition of claim 34 to treat the cancer or a proliferative disease.

36. A human MSLN binder of any one of claims 1-33 or the composition of claim 34 for treatment of cancers or proliferative diseases.

37. Use of a human MSLN binder of any one of claims 1-33 or the composition of claim 34 for the manufacture of a medicament for treating cancer or proliferative disease.

38. A combination therapy for treating a cancer or proliferative disease comprising a human MSLN binder of any one of claims 1-33 or the composition of claim 34 and a therapeutic agent.

39. The combination therapy of claim 38, wherein the therapeutic agent is a chemotherapy agent or a therapeutic antibody.

40. The combination therapy of claim 39, wherein the antibody is an anti-PD1 or anti-PD-L1 antibody.

41. A nucleic acid molecule encoding a human MSLN binder of any one of claims 1-33.

42. An expression vector comprising one or more of the nucleic acid molecules of claim 41.

43. A host cell comprising the expression vector of claim 42.

44. A method for producing a human MSLN binder comprising (a) providing a host cell of claim 43; (b) cultivating the host cell in a medium under conditions suitable for expressing the human MSLN binder; and (c) isolating the human MSLN binder from the medium.

45. A human MSLN binder of any one of claims 1-33 conjugated to a detectable moiety.

46. The human MSLN binder of claim 45, wherein the detectable moiety is detectable by magnetic resonance imaging (MRI) or by X-ray imaging.

47. A human MSLN binder, wherein the human MSLN binder comprises one or two of the modifications relative to the human MSLN binder of any one of claims 1-33 selected from:
(i) a C-terminal amino acid residue K of a heavy chain being removed; and
(ii) a N-terminal amino acid residue E or Q of a VH, a VL, a heavy chain or a light chain being substituted with pyroglutamate.

48. A human MSLN binder, wherein the human MSLN binder is obtainable by expressing the nucleic acid molecule of claim 41 or the expression vector of claim 42 in a host cell.

49. A method for detecting human MSLN on the surface of a cell in a patient comprising administering to the patient a human MSLN binder of claim 45 or 46 and detecting the cells in the patient bound to the human MSLN binder.


SEQUENCE LISTING (41 sequences)

SEQ ID NO: 1 - protein, 622 aa, synthetic construct
  REGION 1..295: note = misc_feature - MPF; megakaryocyte potentiating factor
  REGION 296..622: note = mat_peptide - mature protein
  source 1..622: note = Human Mesothelin (precursor)
MALPTARPLLGSCGTPALGSLLFLLFSLGWVQPSRTLAGETGQEAAPLDGVLANPPNISSLSPRQLLGFPCAEVSGLSTERVRELAVALAQKNVKLSTEQLRCLAHRLSEPPEDLDALPLDLLLFLNPDAFSGPQACTRFFSRITKANVDLLPRGAPERQRLLPAALACWGVRGSLLSEADVRALGGLACDLPGRFVAESAEVLLPRLVSCPGPLDQDQQEAARAALQGGGPPYGPPSTWSVSTMDALRGLLPVLGQPIIRSIPQGIVAAWRQRSSRDPSWRQPERTILRPRFRREVEKTACPSGKKAREIDESLIFYKKWELEACVDAALLATQMDRVNAIPFTYEQLDVLKHKLDELYPQGYPESVIQHLGYLFLKMSPEDIRKWNVTSLETLKALLEVNKGHEMSPQVATLIDRFVKGRGQLDKDTLDTLTAFYPGYLCSLSPEELSSVPPSSIWAVRPQDLDTCDPRQLDVLYPKARLAFQNMNGSEYFVKIQSFLGGAPTEDLKALSQQNVSMDLATFMKLRTDAVLPLTVAEVQKLLGPHVEGLKAEERHRPVRDWILRQRQDDLDTLGLGLQGGIPNGYLVLDLSMQEALSGTPCLLGPGPVLTVLALLLASTLA

SEQ ID NO: 2 - protein, 116 aa, synthetic construct
  source 1..116: note = SV017.47D7.1C2 VH
EVQLVESGGGLVQPGGSLRLSCAATGFTFSSYWMHWVRQGPGKGLVWVSRINSDGTSTVYADSVKGRFTISRDNAKNTLYLQMNSLRAEDTAVYYCARGRGIMTYWGQGTLVTVSS

SEQ ID NO: 3 - protein, 107 aa, synthetic construct
  source 1..107: note = SV017.47D7.1C2 VL
DIQMTQSPSSLSASVGDRVTITCRASQDIGSWLTWYQRKPEKAPKSLIYAASSLQSGVPSRFSGSGSGTDFTLTISSLQPEDFATYYCQQYKSYPLTFGQGAKLEIK

SEQ ID NO: 4 - protein, 5 aa, synthetic construct
  source 1..5: note = SV017.47D7.1C2 VH-CDR1 Kabat
SYWMH

SEQ ID NO: 5 - protein, 17 aa, synthetic construct
  source 1..17: note = SV017.47D7.1C2 VH-CDR2 Kabat
RINSDGTSTVYADSVKG

SEQ ID NO: 6 - protein, 7 aa, synthetic construct
  source 1..7: note = SV017.47D7.1C2 VH-CDR3 Kabat
GRGIMTY

SEQ ID NO: 7 - protein, 11 aa, synthetic construct
  source 1..11: note = SV017.47D7.1C2 VL-CDR1 Kabat
RASQDIGSWLT

SEQ ID NO: 8 - protein, 7 aa, synthetic construct
  source 1..7: note = SV017.47D7.1C2 VL-CDR2 Kabat
AASSLQS

SEQ ID NO: 9 - protein, 9 aa, synthetic construct
  source 1..9: note = SV017.47D7.1C2 VL-CDR3 Kabat
QQYKSYPLT

SEQ ID NO: 10 - protein, 116 aa, synthetic construct
  source 1..116: note = SV018.20B12.1D1 VH
QVQLQESGPGLVKPSETLSLTCTVSGGSISSYYWSWIRQPPGKGLEWIGYIYYSGSTNYNPSLKSRVTISVDTSKNQFSLKLSSVTAADTAVYYCARTGYIPMDVWGQGTTVTVSS

SEQ ID NO: 11 - protein, 107 aa, synthetic construct
  source 1..107: note = SV018.20B12.1D1 VL
EIVLTQSPDFQSVTPKEKVTITCRASQSIGSNLHWYQQKPDQSPKLLIKYVSQSFSGVPSRFSGSGSGTDFTLTINSLEAEDAATYYCHQSRSLPYTFGQGTKLEIK

SEQ ID NO: 12 - protein, 5 aa, synthetic construct
  source 1..5: note = SV018.20B12.1D1 VH-CDR1 Kabat
SYYWS

SEQ ID NO: 13 - protein, 16 aa, synthetic construct
  source 1..16: note = SV018.20B12.1D1 VH-CDR2 Kabat
YIYYSGSTNYNPSLKS

SEQ ID NO: 14 - protein, 8 aa, synthetic construct
  source 1..8: note = SV018.20B12.1D1 VH-CDR3 Kabat
TGYIPMDV

SEQ ID NO: 15 - protein, 11 aa, synthetic construct
  source 1..11: note = SV018.20B12.1D1 VL-CDR1 Kabat
RASQSIGSNLH

SEQ ID NO: 16 - protein, 7 aa, synthetic construct
  source 1..7: note = SV018.20B12.1D1 VL-CDR2 Kabat
YVSQSFS

SEQ ID NO: 17 - protein, 9 aa, synthetic construct
  source 1..9: note = SV018.20B12.1D1 VL-CDR3 Kabat
HQSRSLPYT

SEQ ID NO: 18 - protein, 118 aa, synthetic construct
  source 1..118: note = SV018.45B6.1G1 VH
QVQLLQSGAEVKKPGASVKVSCKASGYTFTTYGISWVRQAPGQGLEWLGWITAYNRNTNYAQKLQGRVTMTTDTSTSTSFMVLRSLRSDDTAVYYCARVGDSDAFDIWGQGTMVTVSS

SEQ ID NO: 19 - protein, 108 aa, synthetic construct
  source 1..108: note = SV018.45B6.1G1 VL
EIVMTQSPATLSLSPGERATLSCRTSQSVFNSFLSWYQQKPGQAPRLLLYGTSTRATGIPARFSGSGSGTDFTLTISSLQPEDFAVYYCQQDYKLPLTFGGGTKVEIK

SEQ ID NO: 20 - protein, 5 aa, synthetic construct
  source 1..5: note = SV018.45B6.1G1 VH-CDR1 Kabat
TYGIS

SEQ ID NO: 21 - protein, 17 aa, synthetic construct
  source 1..17: note = SV018.45B6.1G1 VH-CDR2 Kabat
WITAYNRNTNYAQKLQG

SEQ ID NO: 22 - protein, 9 aa, synthetic construct
  source 1..9: note = SV018.45B6.1G1 VH-CDR3 Kabat
VGDSDAFDI

SEQ ID NO: 23 - protein, 12 aa, synthetic construct
  source 1..12: note = SV018.45B6.1G1 VL-CDR1 Kabat
RTSQSVFNSFLS

SEQ ID NO: 24 - protein, 7 aa, synthetic construct
  source 1..7: note = SV018.45B6.1G1 VL-CDR2 Kabat
GTSTRAT

SEQ ID NO: 25 - protein, 9 aa, synthetic construct
  source 1..9: note = SV018.45B6.1G1 VL-CDR3 Kabat
QQDYKLPLT

SEQ ID NO: 26 - protein, 116 aa, synthetic construct
  source 1..116: note = SV017.13B12.1E1 VH
EVQLVESGGGLVQPGGSLRLSCAATGFTFSSYWMHWVRQGPGKGLVWVSRINSDGTSTVYADSVKGRFTISRDNAKNTLHLQMNSLRAEDTAVYYCARGRGIMTYWGQGTLVTVSS

SEQ ID NO: 27 - protein, 116 aa, synthetic construct
  source 1..116: note = SV017.50G3.1B1 VH
EVQLVESGGGLVQPGESLRLSCAATGFTFSSYWMHWVRQGPGKGLVWVSRINSDGTSTVYADSVKGRFTISRDNAKNTLFLQMNSLRAEDTAVYYCARGRGIMTYWGQGTLVTVSS

SEQ ID NO: 28 - protein, 116 aa, synthetic construct; identical to SEQ ID NO: 2
  source 1..116: note = SV017.51F4.1A1 VH

SEQ ID NO: 29 - protein, 330 aa, synthetic construct [sequence omitted]

SEQ ID NO: 30 - protein, 330 aa, synthetic construct
  source 1..330: note = Human IgG1 HC Constant domain (E233A/L235A)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPALAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 330 aa, synthetic construct
  source 1..330: note = Human IgG1 HC Constant domain (L234A L235A D265S)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 330 aa, synthetic construct
  source 1..330: note = Human IgG1 HC Constant domain (L234A L235A P329G)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - protein, 330 aa, synthetic construct
  source 1..330: note = Human IgG1 HC Constant domain (L235E)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 330 aa, synthetic construct
  source 1..330: note = Human IgG1 HC Constant domain (D265A)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - protein, 330 aa, synthetic construct
  source 1..330: note = Human IgG1 HC Constant domain (D265A N297G)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 36 - protein, 330 aa, synthetic construct
  VARIANT 180: note = Xaa is any amino acid other than N
  source 1..330: note = Human IgG1 HC Constant domain (N297X)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYXSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 37 - protein, 330 aa, synthetic construct
  source 1..330: note = Human IgG1 HC Constant domain (N297A/D356E/L358M)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 38 - protein, 107 aa, synthetic construct [sequence omitted]

SEQ ID NO: 39 - protein, 621 aa, synthetic construct
  REGION 1..294: note = misc_feature - MPF; megakaryocyte potentiating factor
  REGION 295..621: note = mat_peptide - mature protein
  source 1..621: note = Rhesus mesothelin precursor
MALPMARPLSGSCGTPAVGSLLFLLFSLGWVQPSRVLAGETRQEAAPLDGILTNAPDIASLSPRQLLGFTCVEVSGLSTELVQELAVALGQKNVKLSAEQLRCLAHRLSEPPEDLALPLDLLLFLNPDAFSGPQACTHFFSRVAKANVDLLPRGAPERQRLLPAALTCWGVRGSLLSEADVRALGGLACDLPGCFVAESAEVVLPRLVRCLGPLDQDQQEAARAALQRGGPPYGPPSTWSISTLDDLQSLLPVLGQPVIHSIPKGILAAWRQRSSRDPSWQQPEQTVLRPRFRRDVERTTCPPEKEVHEIDESLIFYKKRELEACVDAALLAAQMDRVDAIPFTYEQLDVLKHKLDELYPQGYPESVIRHLGHLFLKMSPEDIRKWNVTSLETLKALLKVSKGHEMSAQVATLIDRVVVGRGQLDKDTVDTLTAFCPGCLCSLSPERLSSVPPSVIGAVRPQDLDTCGPRQLDVLYPKARLAFQNMSGSEYFVKIRPFLGGAPTEDVKALSQQNVSMDLATFMKLRREAVLPLTVAEVQKLLGPHVEGLKVEEQHSPVRDWILKQRQDDLDTLGLGLQGGIPNGYLILDLSVREALSGTPCLLGPGPVLTVLALLLASTLA

SEQ ID NO: 40 - protein, 449 aa, synthetic construct
  source 1..449: note = MORAB-009 Heavy chain
QVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGSGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 41 - protein, 213 aa, synthetic construct
  source 1..213: note = MORAB-009 light chain
DIELTQSPAIMSASPGEKVTMTCSASSSVSYMHWYQQKSGTSPKRWIYDTSKLASGVPGRFSGSGSGNSYSLTISSVEAEDDATYYCQQWSKHPLTFGSGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC